(19) United States
(12) Patent Application Publication
(10) Pub. No.: US 2018/0153889 A1
(43) Pub. Date: Jun. 7, 2018

(54) MODULATION OF CELL FATES AND ACTIVITIES BY PHTHALAZINEDIONES

(71) Applicant: Bach Pharma, Inc., North Andover, MA (US)

(72) Inventors: Mark O. Henry, North Andover, MA (US); William S. Lynn, Hillborough, NC (US)

(21) Appl. No.: 15/883,055

(22) Filed: Jan. 29, 2018

Related U.S. Application Data
(63) Continuation-in-part of application No. 12/565,579, filed on Sep. 23, 2009, now abandoned.
Application No. 12/565,579 is a continuation-in-part of application No. 13/764,365, filed on Feb. 11, 2013.
Application No. 13/764,365 is a continuation of application No. 13/537,900, filed on Jun. 29, 2012, now abandoned.
Application No. 13/537,900 is a continuation of application No. 13/294,634, filed on Nov. 11, 2011, now abandoned.
Application No. 13/294,634 is a continuation of application No. 13/074,784, filed on Mar. 29, 2011, now abandoned.
Application No. 13/074,784 is a continuation of application No. 12/044,716, filed on Mar. 7, 2008, now abandoned.
Application No. 12/044,716 is a continuation of application No. 11/699,853, filed on Jan. 30, 2007, now abandoned.
Application No. 11/699,853 is a division of application No. 11/199,394, filed on Aug. 8, 2005, now Pat. No. 7,326,690.
Application No. 11/199,394 is a continuation-in-part of application No. 10/283,647, filed on Oct. 30, 2002, now Pat. No. 6,953,799.
(60) Provisional application No. 61/150,581, filed on Feb. 6, 2009.
(60) Provisional application No. 61/099,456, filed on Sep. 23, 2008.

(51) Int. Cl. A61K 31/502; A61K 31/195; A61K 31/385; A61K 31/4743; A61K 31/55; A61K 31/19; A61K 31/573; A61K 31/65; A61K 38/06; A61K 45/06; A61K 31/56

(52) U.S. Cl. CPC A61K 31/502; A61K 31/195; A61K 31/385; A61K 31/4743; A61K 31/55; A61K 2300/00; A61K 31/573; A61K 31/65; A61K 38/063; A61K 45/06; A61K 31/56; A61K 31/19

(57) ABSTRACT

Phthalazinediones that function as intracellular redox modulators in the redox therapy of certain stressed cells are provided. By buffering aberrant redox states, phthalazinediones enable cellular processes essential for survival and augment medical treatments. The phthalazinediones of the invention can modulate functions related to cell growth, differentiation, activity, or death, to correct aberrations and restore homeostasis, and can serve as adjunctive therapy in treating various disease conditions.

### RELATED APPLICATIONS

This application is a continuation-in-part of U.S. application Ser. No. 12/565,579, filed on Sep. 23, 2009, which claims the benefit of U.S. Provisional Application No. 61/150,581, filed on Feb. 6, 2009 and U.S. Provisional Application No. 61/099,456, filed on Sep. 23, 2008, and is a continuation-in-part of U.S. application Ser. No. 13/764,365, filed Feb. 11, 2013, which is a continuation of U.S. application Ser. No. 13/537,900, filed Jun. 29, 2012, which is a continuation of U.S. application Ser. No. 13/294,634, filed Nov. 11, 2011, which is a continuation of U.S. application Ser. No. 13/074,784, filed Mar. 29, 2011, which is a continuation of U.S. application Ser. No. 12/044,716, filed Mar. 7, 2008, which is a continuation of U.S. application Ser. No. 11/699,853, filed Jan. 30, 2007, which is a divisional of U.S. application Ser. No. 11/199,394, filed Aug. 8, 2005, which is a continuation-in-part of U.S. application Ser. No. 10/283,647, filed Oct. 30, 2002, which are incorporated herein by reference in their entirety the entire teachings of which are incorporated herein by reference.

### BACKGROUND OF THE INVENTION

Current medical treatments generally focus on the disease and strive to eliminate the inciting agent or the symptoms, often injuring healthy tissue in the process. In healthy cells, a balance of redox reactions maintains a physiologically appropriate environment for various cellular functions related to growth, differentiation, activity, and death. The proper coordination of such functions ensures homeostasis and the health of cells. Research has shown that alterations in cellular redox status affect activities such as cellular signaling, suggesting that altering the cellular redox status could also affect cellular activation, which results from certain cellular signals (U.S. Pat. No. 5,994,402). Altering the intracellular redox state by upregulating Nrf2, thereby increasing cells of glutathione (GSH), an endogenous “redox agent,” has also been shown to protect cells from certain injury and to promote their survival (U.S. Pat. No. 5,994,402), again suggesting a link between alterations in the cellular redox state and cellular functions.

Stresses that perturb a cell's redox status may be internal or external. For example, a genetic mutation may produce defective protein products that function abnormally or not at all. For example, the proteins may fold improperly. These defective proteins could disrupt certain cellular processes, including redox reactions. Cellular redox reactions may also be disrupted by microbes, toxins, allergens, or other agents external to the cell. The stress could also be due to heavy metal intoxication, metabolic syndrome or the deregulation of Nrf2 transcription factors. The external stress could trigger defensive responses that leave the cell's redox system depleted and unstable.

An imbalanced redox state, even if not the cause of a particular disease condition, may facilitate that condition by providing an “unhealthy” environment in which necessary cellular functions become impaired. Cellular redox status may become impaired in numerous disease conditions. Under the stress of a disease state, the rate of redox reactions increases or decreases as needed by the cell. However, significant or prolonged deviations in the intracellular redox status disable cellular processes, including defense mechanisms. When such cellular functions are impaired, the survival of the cell becomes uncertain. Under such stressed environments these disturbances causes the increased production of reactive oxygen species (ROS) and/or reactive nitrogen species (RNS). Maintenance of the proper redox status is thus critical to the fate of the cell.

To counter and correct disturbances in the redox status, cells require agents that can modulate redox imbalances, to facilitate reduction or oxidation reactions as appropriate. Agents currently available for correcting redox imbalances are inadequate in that they are labile, quickly oxidized, or unable to translocate to the proper region of the cell. Examples of such exogenous redox agents include cysteine, reduced lipoates or thiols, glucocorticoids, and other antioxidants. Redox agents that remain stable, active, and functional in the cellular environment are necessary.

Although their role in modulating intracellular redox status was not recognized, phthaloylhydrazide, phthalazinedione, and phthalazine derivatives have been described as having anti-inflammatory, anti-cancer, and anti-hypoxic effects (U.S. Pat. Nos. 6,686,347; 6,489,326; 5,874,444; 5,543,410; 5,512,573; 4,250,180). However, toxicity and the lack of pharmacological activity of certain phthaloylhydrazides, including 2,3-dihydrophthalazine-1,4-dione and 5-amino-2,3-dihydrophthalazine-1,4-dione, were noted (U.S. Pat. Nos. 6,489,326; 5,543,410; 5,512,573). Luminol, also known as o-aminophthaloylhydrazide, 3-aminophthalhydrazide, 5-aminophthaloylhydrazide, or 5-amino-2,3-dihydro-1,4-phthalazinedione, was considered toxic and used in photothermographic imaging, chemiluminescent assays and labeling of cellular structures, detection of copper, iron, peroxides, or cyanides, and forensic science to detect traces of blood (U.S. Pat. Nos. 5,279,940; 4,729,950; Merck Index, 13th ed. (2001), monograph no. 5622).

Nonetheless, the compound 5-aminophthaloylhydrazide was identified for use in treating inflammatory conditions such as ulcerative colitis, Crohn's disease, diffuse sclerosis, diarrhea, proctitis, hemorrhoids, anal fissures, dyspepsia, intestinal infection, Alzheimer's disease, osteoarthritis, macular degeneration, and proctosigmoiditis (U.S. Pat. Nos. 5,874,444; 5,543,410; EP 617024; RU2211036), as well as for use in treating psoriasis, infarct, and transplant rejection (U.S. Pat. Nos. 6,489,326; 5,512,573). Other phthaloylhydrazide derivatives identified as having pharmacological activity include 2,3-dihydrophthalazine-1,4-dione, 2-amino-1,2,3,4-tetrahydrophthalazine-1,4-dione sodium salt dihydrate, 4-aminophthaloylhydrazide, 4,5-aminophthaloylhydrazide, and 4,5-methylaminophthaloylhydrazide (U.S. Pat. Nos. 6,489,326; 5,512,573; RU 2113222).

Phthalazinedione compounds, including luminol, have also been described as an inhibitor of poly (ADP-ribose) polymerase, an enzyme that responds to DNA damage (U.S. Pat. Nos. 5,874,444; 5,719,151; 5,633,282), and for treating conditions involving the functions of poly (ADP-ribose) polymerase (U.S. Pat. Nos. 5,874,444; 5,719,151; 5,633,282). A method of manufacturing the sodium salt of 5-amino-2,3-dihydrophthalazine-1,4-dione and its pharmaceutical use for immunomodulation, inflammation, and anti-oxidant treatment have been described (U.S. Pat. No. 6,489,326; RU 2222327).

Cells normally maintain a balance between protein synthesis, folding, trafficking, aggregation, and degradation. This balance is referred to as protein homeostasis and the balance is maintained by utilizing sensors and networks of pathways. Human loss of function diseases are often the result of a disruption of normal protein homeostasis, typically caused by a mutation in a given protein that compromises its cellular folding, leading to efficient degradation. There is therefore insufficient function because the concentration of the mutant protein is exceedingly low.

There are at least 40 distinct lysosomal storage diseases (LSDs) resulting from the deficient function of a single mutated enzyme in the lysosome, leading to accumulation of corresponding substrate(s). Futerman et al., Nat Rev Mol Cell Biol 5: 554-565, 2004; Sawkar et al., Cell Mol Life Sci 63: 1179-1192, 2006. Currently, LSDs are treated by enzyme replacement therapy. However, this can be challenging because the endocytic system has to be utilized to get the recombinant enzyme into the lysosome. Desnick et al., Nat Rev Genet. 3: 954-966, 2002.

The cellular maintenance of protein homeostasis, or proteostasis, refers to controlling the conformation, binding interactions, location and concentration of individual proteins making up the proteome. Since proteins play a central role in the physiology of all organisms, loss of the normal balance between proper protein folding, localization and degradation influences or causes numerous diseases. Albanese, V., et al., Cell 124: 75-88, 2006; Brown et al., J Clin Invest 99: 1432-1444, 1997; Cohen et al., Nature 426: 905-909, 2003; Deuerling et al., Crit Rev Biochem Molec Biol 39: 261-277, 2004; Horwich et al., Encyclopedia Biol Chem 1: 393-398, 2004; Imai et al., Cell Cycle 2: 585-589, 2003; Kaufman, J Clin Invest 110: 1389-1398, 2002; Ron et al., Nat Rev Mol Cell Biol 8: 519-529, 2007; Young et al., Nat Rev Mol Cell Biol 5: 781-791, 2004. Protein folding in vivo is accomplished through interactions between the folding polypeptide chain and macromolecular cellular components, including multiple classes of chaperones and folding enzymes, which minimize aggregation. Wiseman et al., Cell 131: 809-821, 2007. Metabolic enzymes also influence cellular protein folding efficiency because the organic and inorganic solutes produced by a given compartment effect polypeptide chain salvation through non-covalent forces, including the hydrophobic effect, that influences the physical chemistry of folding. Metabolic pathways also produce small molecule ligands that can bind to and stabilize the folded state of a specific protein, enhancing folding by shifting folding equilibria. Fan et al., Nature Med., 5, 112 (January 1999); Hammarstrom et al., Science 299, 713 (2003). Whether a given protein folds in a certain cell type depends on the distribution, concentration, and subcellular localization of chaperones, folding enzymes, metabolites and the like. Wiseman et al., Cell 131: 809-821, 2007.

Loss-of-function diseases are often caused by the inability of a mutated protein to fold properly within and traffic through the secretory pathway, leading to extensive endoplasmic reticulum (ER) associated degradation (ERAD) and thus to a lowered concentration of the protein in its destination environment. Brodsky, Biochem J 404: 353-363, 2007; Brown et al., J Clin Invest 99: 1432-1444, 1997; Cohen et al., Nature 426: 905-909, 2003; Moyer et al., Emerg Ther Targets 5: 165-176, 2001; Sawkar et al, Cell Mol Life Sci 63: 1179-1192, 2006a; Schroeder et al., Ann Rev Biochem 74: 739-789, 2005; Ulloa-Aguirre et al., Traffic 5: 821-837, 2004; Wang et al., Cell 127: 803-815, 2006; Wiseman et al., Cell 131: 809-821, 2007. Lysosomal storage diseases (LSDs) are loss-of-function diseases often caused by extensive ERAD of a mutated lysosomal enzyme instead of proper folding and lysosomal trafficking. Fan, Front Biotechnol Pharm 2: 275-291, 2001; Fan et al., Nat Med 5: 112-115, 1999; Futerman et al., Nat Rev Mol Cell Biol 5: 554-565, 2004; Sawkar et al., Chem Biol 12: 1235-1244, 2005; Sawkar et al., Proc Natl Acad Sci USA 99:15428-15433, 2002; Sawkar et al., Cell Mol Life Sci 63: 1179-1192, 2006a; Sawkar et al., ACS Chem Biol 1: 235-251, 2006b; Schmitz et al., Int J Biochem Cell Biol 37: 2310-2320, 2005; Yu et al., J Med Chem 50: 94-100, 2007b; Zimmer et al., J Pathol 188: 407-414, 1999. They are characterized by substrate accumulation, which typically arises when the activity of a mutated lysosomal enzyme drops below ≈10% of normal. Conzelmann et al., Dev Neurosci 6: 58-71, 1984; Schueler et al., J Inherit Metab Dis 27: 649-658, 2004. LSDs are now treated by replacing the damaged enzyme with a wild type recombinant version that utilizes the endocytic pathway to reach the lysosome. Futerman et al., Nat Rev Mol Cell Biol 5: 554-565, 2004; Beutler et al., Proc Natl Acad Sci USA 74: 4620-4623, 1977; Brady, Ann Rev Med 57: 283-296, 2006 Enzyme replacement therapy fails for neuropathic LSDs because the recombinant enzyme does not cross the blood brain barrier. Sawkar et al, Cell Mol Life Sci 63: 1179-1192, 2006a. Many of mutated lysosomal enzymes that misfold and are degraded by ERAD can fold and exhibit partial activity under appropriate conditions, such as when the cells are grown at a lower permissive temperature. Futerman et al., Nat Rev Mol Cell Biol 5: 554-565, 2004; Sawkar et al., ACS Chem Biol 1: 235-251, 2006b. The challenge for most mutated glycolipid processing enzymes is to fold in the neutral pH environment of the ER, distinct from that of the acidic environment of the lysosome. Sawkar et al., ACS Chem Biol 1: 235-251, 2006b.

New strategies are needed to develop effective therapies for diseases related to intracellular protein misfolding and altered protein trafficking which can lead to loss of function diseases such as lysosomal storage disease and neuropathic lysosomal storage disease, or gain of function disease such as age-onset related disease, e.g., age-related macular degeneration, inclusion body myositosis, type II diabetes, amyotrophic lateral sclerosis, Alzheimer's disease, Huntington's disease or Parkinson's disease. Since current treatments are limited to compounds approved for enzyme replacement therapy or substrate reduction therapy, a need exists in the art for new therapeutic approaches to treat protein loss of function diseases or gain of function diseases related to dysfunction in protein homeostasis.

Inflammation stress is increasingly regarded as a key process underlying metabolic diseases in obese individuals. Particularly, obese adipose tissue shows features characteristic of active local inflammation. Nishimura et al., Nat Med 15: 914-921, 2009. These stresses are similarly related to the increased production of ROS and RNS. Nishimura et al. is hereby incorporated by reference in its entirety herein.

It has been demonstrated that N-acetyl cystein or mannitol had a potentiating effect on the chelating ability of monoisoamyl-2,3-dimercaptosuccinate (MiADMS). Tandon et al., Toxicology Letters 145: 211-217, 2003. Tandon et al. is hereby incorporated by reference in its entirety herein. For example the treatment of cadmium intoxication with MiADMS was demonstrated to be improved when coadministered with N-acetyl cysteine (NAC) and even more improved when the MiADMS is combined with mannitol.

### SUMMARY OF THE INVENTION

The present invention provides suitable means for modulating the effects of metabolic syndrome, heavy metal intoxication, protein homeostasis and Nrf2 transcription factor without the above drawbacks.

Phthalazinediones of the invention may be used to modulate redox imbalances and to support a patient's body in a variety of disease states and in treating metabolic distress, inflammation, infectious conditions, neurological disorders, immune disorders, proliferative diseases, and senescence. The phthalazinediones may also be used in conjunction with standard treatment methods such as chemotherapy, radiation, nutrition, pharmaceutical treatment, and surgery.

In some aspects, the disclosure provides phthalazinedione compounds comprising N-ethanoyl-5-amino-2,3-dihydrophthalazine-1,4-dione or 5-(hexanoyl oxyamino)-2,3-dihydrophthalazine-1,4-dione.

In some aspects, the disclosure provides therapeutic formulations comprising N-ethanoyl-5-amino-2,3-dihydrophthalazine-1,4-dione or 5-(hexanoyl oxyamino)-2,3-dihydrophthalazine-1,4-dione.

In some aspects, the disclosure provides pharmaceutical compositions comprising N-ethanoyl-5-amino-2,3-dihydrophthalazine-1,4-dione or 5-(hexanoyl oxyamino)-2,3-dihydrophthalazine-1,4-dione, or the pharmaceutically acceptable salt, ester, solvate, hydrate, metabolite, enantiomer, isomer, tautomer, amide, prodrug, or free base; and one or more pharmaceutically acceptable carriers.

In some embodiments, the pharmaceutical compositions include an amino acid, antibiotic, antiviral agent, anti-inflammatory agent, antioxidant, immunomodulator, reductant, oxidative protector, steroid, vitamin, or a combination thereof. In some embodiments, the pharmaceutical composition comprises a pharmaceutically acceptable form selected from the group consisting of tablet, capsule, granule, powder, solution, suspension, microsphere, liposome, colloid, lyophilized composition, gel, lotion, ointment, cream, spray, and suppository.

In some aspects, the disclosure provides methods of administering to a subject a pharmaceutical composition as described herein.

In some embodiments of any aspect described herein, the pharmaceutical composition is administered to the subject by a means selected from the group consisting of intravenous, intramuscular, intraperitoneal, subcutaneous, oral, nasal, mucosal, transdermal, parenteral, vaginal, and rectal. The pharmaceutical composition may be administered to the subject in combination with a standard treatment selected from the group consisting of radiation, chemotherapy, laser therapy, surgery, medication, and nutrition.

In some embodiments, the pharmaceutical composition is administered to the subject to treat metabolic distress, inflammatory conditions, infectious conditions, neurological disorders, immune disorders, proliferative diseases, or cellular senescence.

In some embodiments, the pharmaceutical composition is administered to the subject in a dose comprising the N-ethanoyl-5-amino-2,3-dihydrophthalazine-1,4-dione in an amount of about 0.01 mg/kg to about 100.0 mg/kg, in an amount of about 0.05 mg/kg to about 50.0 mg/kg, or in an amount of about 0.1 mg/kg to about 10.0 mg/kg of body weight. In some embodiments, the pharmaceutical composition is administered to the subject in a dose comprising the N-ethanoyl-5-amino-2,3-dihydrophthalazine-1,4-dione in an amount of about 1.0 mg per day to about 10,000.0 mg, in an amount of about 50.0 mg per day to about 5000.0 mg per day, or in an amount of about 100.0 mg per day to about 1000.0 mg per day. In some embodiments, the pharmaceutical composition is administered to the subject in a dose comprising the N-ethanoyl-5-amino-2,3-dihydrophthalazine-1,4-dione in an amount of about 1.0 mg, 10.0 mg, 50.0 mg, 100.0 mg, 200.0 mg, 300.0 mg, 400.0 mg, 500.0 mg, 600.0 mg, 700.0 mg, 800.0 mg, 900.0 mg, 1000.0 mg, 2000.0 mg, 3000.0 mg, 4000.0 mg, 5000.0 mg, 6000.0 mg, 7000.0 mg, 8000.0 mg, 90000 mg, or 10,000.0 mg per day.

In some aspects, the disclosure provides methods of modulating the inflammatory manifestations of metabolic syndrome. The methods comprise administering a redox support therapy to a subject in need thereof, wherein the redox support therapy comprises N-ethanoyl-5-amino-2,3-dihydrophthalazine-1,4-dione or 5-(hexanoyl oxyamino)-2,3-dihydrophthalazine-1,4-dione.

In some aspects, the disclosure provides methods of modulating the effects of heavy metal intoxication. The methods comprise administering a chelation therapy to a subject in need thereof, wherein the chelation therapy comprises MiADMS and N-ethanoyl-5-amino-2,3-dihydrophthalazine-1,4-dione or 5-(hexanoyl oxyamino)-2,3-dihydrophthalazine-1,4-dione.

In some aspects, the disclosure provides methods of modulating the effects of Zinc intoxication. The methods comprise administering a chelation therapy to a subject in need thereof, wherein the chelation therapy comprises N-ethanoyl-5-amino-2,3-dihydrophthalazine-1,4-dione or 5-(hexanoyl oxyamino)-2,3-dihydrophthalazine-1,4-dione, and a second agent, wherein the second agent is selected from the group consisting of CaEDTA, TPEN, and pyrithione.

In other aspects, the disclosure provides methods of modulating the effects of Copper intoxication. The methods comprise administering a chelation therapy to a subject in need thereof, wherein the chelation therapy comprises N-ethanoyl-5-amino-2,3-dihydrophthalazine-1,4-dione or 5-(hexanoyl oxyamino)-2,3-dihydrophthalazine-1,4-dione, and a second agent, wherein the second agent is selected from the group consisting of CaEDTA, TPEN, and pyrithione.

In some aspects, the disclosure provides methods of modulating the effects of iron intoxication. The methods comprise administering a chelation therapy to a subject in need thereof, wherein the chelation therapy comprises N-ethanoyl-5-amino-2,3-dihydrophthalazine-1,4-dione or 5-(hexanoyl oxyamino)-2,3-dihydrophthalazine-1,4-dione, and a second agent.

In some aspects, the disclosure provides methods for treating a condition characterized by dysfunction in protein homeostasis in a patient in need thereof. The methods comprise administering to the patient a proteostasis regulator in an amount effective to improve or restore protein homeostasis, and to reduce or eliminate the condition in the patient or to prevent its occurrence or recurrence, wherein the proteostasis regulator is N-ethanoyl-5-amino-2,3-dihydrophthalazine-1,4-dione or 5-(hexanoyl oxyamino)-2,3-dihydrophthalazine-1,4-dione.

### DETAILED DESCRIPTION

The present invention focuses on the patient, to enable self-repair mechanisms by supporting the patient's body in controlling or stabilizing its cellular functions without toxic side effects. The methods and compositions of the invention comprise phthalazinedione compounds that buffer intracellular reduction and oxidation (redox) reactions and thereby modulate cellular functions of growth, differentiation, activity, and death in various disease states.

In one aspect the present invention describes the use of phthalazinedione compounds as redox support therapy in treating diseases or disorders involving impaired or aberrant intracellular redox states wherein ROS and/or RNS are produced. By buffering redox imbalances, phthalazinediones reversibly and selectively modulate cellular functions, e.g., upregulating mitochondrial aerobic metabolism when a cell under stress needs energy for defense or repair, or downregulating metabolism when the stressed cell is overactive. Phthalazinediones can modulate cellular processes such as proliferation, secretion, differentiation, transformation, migration, and apoptosis, without toxic side effects on healthy cells.

Under any stress, intracellular redox status is inevitably impaired as aerobic metabolism is necessarily overworked. Any stress to the cell, especially if prolonged, will deplete the cell of endogenous redox agents, including thiols, glutathione, thioredoxins, iron-sulfur proteins, cysteine, and thiol proteins, as well as redox-sensitive proteins such as catalase. Chronic stress leads to cellular and organelle thiol deficiencies, as blood cysteine is limited. In turn, since many cellular pathways are controlled by or depend on intracellular redox activities, thiol deficiencies lead rapidly to impaired energy production, with increased oxidant production and progressive mitochondrial and cell death.

In mitochondrial aerobic metabolism, electron flow is fragile and easily perturbed by oxidant stresses. Under stress, the cell must rapidly increase both the electron flow and the subsequent membrane proton (H⁺) gradient. However, electron flow and proton gradient may fail if overactivated or stressed. Electrons are then diverted directly to oxygen (O₂), producing toxic superoxide (O₂⁻), while the proton gradient declines, hindering ATP production. Moreover, under oxidant stress, mitochondrial membrane channels and permeability pores become oxidized, which distorts the channels and opens the pores. Consequently, protons, substrate anions, glutamate, reductants, cytochrome c, and nucleotides all leak through the distorted channels and opened pores, leaving the mitochondrion and cell deficient in essential substances, energy, and redox status.

With prolonged thiol deficiencies, replacement therapy with available thiols is difficult and usually inadequate. Cysteine and other reduced thiols are labile and rapidly oxidized to toxic metabolites in the presence of oxygen. Most antioxidants, which dissipate oxygen-based oxidants, are unable to penetrate to the electron-transporting inner mitochondrial membrane to modulate the iron-sulfur protein mediated electron flow in mitochondrial Complex III or to stabilize disulfide cross-linkages that control permeability of the mitochondrial megapores and channels. Antioxidants also cannot supply the cysteine required in the manufacture of most proteins or the energy required to combat chronic stresses or repair cellular damages.

The phthalazinediones, as described herein for use in the methods and compositions, have been found to be suitable alternatives for modulating the redox imbalances.

In general, a therapeutically effective amount of a phthalazinedione of the present invention that is sufficient to ameliorate disease symptoms will depend on the acuteness of the disease, the particular redox status or deficiency of the patient, the developmental condition of the stressed cell, and also the state of oxidation of the phthalazinedione, but will be in the range of about 0.01-100.0 mg per kg of body weight or about 1.0-10,000.0 mg per day, e.g., administered in amounts of 1.0, 10.0, 50.0, 100.0, 200.0, 300.0, 400.0, 500.0, 600.0, 700.0, 800.0, 900.0, 1000.0, 2000.0, 3000.0, 4000.0, 5000.0, 6000.0, 7000.0, 8000.0, 9000.0, or 10,000.0 mg.

The phthalazinedione compounds of the present invention are preferably incorporated into pharmaceutical forms suitable for administration by oral, nasal, mucosal, vaginal, rectal, transdermal, or parenteral routes, including subcutaneous, intramuscular, intravenous, and intraperitoneal, e.g., tablet, capsule, granule, powder, solution, suspension, microsphere, liposome, colloid, lyophilized composition, gel, lotion, ointment, cream, spray, and suppository, and preferably include pharmaceutically acceptable excipients, carriers, adjuvants, diluents, or stabilizers as is well known to the skilled in the art. Suitable phthalazinediones have a purity of at least 90%. More preferably, the purity is at leas 95% or 98.6% or 99%. All ranges within these specific percentages are also contemplated in the present invention. These phthalazinediones have been described in co-pending U.S. Provisional Patent Application No. 61/150,581 which is hereby incorporated by reference in its entirety.

The phthalazinedione may be a derivative compound containing a substituent that enhances the activity, stability, or other property of the compound. Such a derivative compound may be an amino phthalazinedione or a phthalazinedione comprising a haloamino, alkylamino, acylamino, alkanolamino, alkenylamino, alkoxyamino, haloalkylamino, allylamino, or sulfhydrylamino (thiolamino or mercaptoamino) group or other substituents that confer a preferred function on the compound. Furthermore, the phthalazinedione may be a bromoamino, chloroamino, fluoroamino, iodoamino, methylamino, ethylamino, propylamino, isopropylamino, methanoylamino(formylamino), ethanoylamino(acetylamino), propanoylamino, hydroxylamino, carboxylamino, methanol amino, ethanolamino, propanolamino, methenylamino, ethenylamino, propenylamino, methoxyamino, ethoxyamino, propoxyamino, or dimethylamino derivative.

Examples of such phthalazinedione derivatives include, but are not limited to, 5-amino-2,3-dihydrophthalazine-1,4-dione (luminol), 6-amino-2,3-dihydrophthalazine-1,4-dione (isoluminol), 5-amino-2,3-dihydrophthalazine-1,4-dion-8-yl (luminyl), N-bromo-5-amino-2,3-dihydrophthalazine-1,4-dione, N-chloro-5-amino-2,3-dihydrophthalazine-1,4-dione, N-fluoro-5-amino-2,3-dihydrophthalazine-1,4-dione, N-iodo-5-amino-2,3-dihydrophthalazine-1,4-dione, N-methyl-5-amino-2,3-dihydrophthalazine-1,4-dione, N-ethyl-5-amino-2,3-dihydrophthalazine-1,4-dione, N-propyl-5-amino-2,3-dihydrophthalazine-1,4-dione, N-isopropyl-5-amino-2,3-dihydrophthalazine-1,4-dione, N-methanoyl-5-amino-2,3-dihydrophthalazine-1,4-dione, N-ethanoyl-5-amino-2,3-dihydrophthalazine-1,4-dione, N-propanoyl-5-amino-2,3-dihydrophthalazine-1,4-dione, N-hydroxyl-5-amino-2,3-dihydrophthalazine-1,4-dione, N-carboxyl-5-amino-2,3-dihydrophthalazine-1,4-dione, N-methanol-5-amino-2,3-dihydrophthalazine-1,4-dione, N-ethanol-5-amino-2,3-dihydrophthalazine-1,4-dione, N-propanol-5-amino-2,3-dihydrophthalazine-1,4-dione, N-methenyl-5-amino-2,3-dihydrophthalazine-1,4-dione, N-ethenyl-5-amino-2,3-dihydrophthalazine-1,4-dione, N-propenyl-5-amino-2,3-dihydrophthalazine-1,4-dione, N-methoxy-5-amino-2,3-dihydrophthalazine-1,4-dione, N-ethoxy-5-amino-2,3-dihydrophthalazine-1,4-dione, N-propoxy-5-amino-2,3-dihydrophthalazine-1,4-dione, N,N-dimethyl-5-amino-2,3-dihydrophthalazine-1,4-dione, N-acetylcysteine-5-amino-2,3-dihydrophthalazine-1,4-dione, and N-acetylglutathione-5-amino-2,3-dihydrophthalazine-1,4-dione. Enantiomers, isomers, tautomers, esters, amides, salts, solvates, hydrates, analogues, metabolites, free bases, or prodrugs of the phthalazinedione or its derivative are also contemplated by the invention.

In some embodiments, a phthalazinedione derivative is a N-ethanoyl-5-amino-2,3-dihydrophthalazine-1,4-dione or 5-(acetoxyamino)-2,3-dihydrophthalazine-1,4-dione, also referred to as Monosodium Luminol Ester. The phthalazinedione has the following chemical structure:

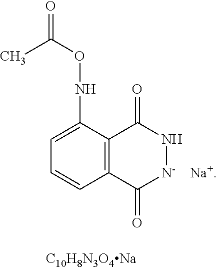

A N-ethanoyl-5-amino-2,3-dihydrophthalazine-1,4-dione exhibits various benefits over a phthalazinedione including having superior absorption characteristics and a faster delivery of the electron transfer capability to the site of the redox imbalance.

In other embodiments, a phthalazinedione derivative is a 5-(hexanoyl oxyamino)-2,3-dihydrophthalazine-1,4-dione, also referred to as Monosodium Luminol Hexonyl, and having the following chemical structure:

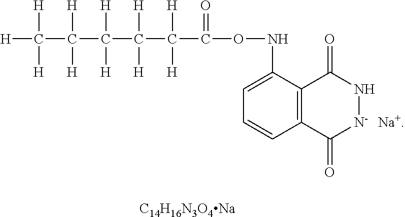

Monosodium luminol hexonyl and monosodium lumino hexanoyl are used interchangeably herein. A 5-(hexanoyl oxyamino)-2,3-dihydrophthalazine-1,4-dione exhibits various benefits over a phthalazinedione including having superior hydrophilic characteristics and a faster delivery of electrons to the site of the redox imbalance. The hydrophobic (water-fearing) side change increases solubility in cell membranes, specifically the fatty acid transporters in the mitochondria.

The phthalazinedione derivatives described herein exhibit increased activity and physiological accessibility as compared to previously known compounds. It is generally understood that during synthesis of these molecular derivatives of Luminol with the functional organic groups at the sites of the NH²⁺ charge, the functional organic groups essentially block or compete with the charge. The derivatives demonstrate increased solubility, and competitive binding to cationic channels and amine neurotransmitters (e.g., dopamine, serotonin, and adrenaline) is increased.

In some embodiments, a pharmaceutical composition comprises one or more phthalazinedione derivatives. The pharmaceutical composition may include N-ethanoyl-5-amino-2,3-dihydrophthalazine-1,4-dione, 5-(hexanoyl oxyamino)-2,3-dihydrophthalazine-1,4-dione, or combinations thereof. In some aspects, the phthalazinedione derivative includes a pharmaceutically acceptable salt, ester, solvate, hydrate, metabolite, enantiomer, isomer, tautomer, amide, prodrug, or free base. In certain aspects, the pharmaceutical composition includes one or more phthalazinedione derivatives and one or more pharmaceutically acceptable carriers.

In an embodiment of the invention, phthalazinediones can be used to either facilitate or inhibit electron flow in mitochondria, and thus control ATP production. For example, in vitro, at the low dose of 20-50 μM, amino phthalazinediones facilitate electron flow at mitochondrial Complex III, thereby increasing ATP production, DNA synthesis, and cell cycling, for cell growth. At an intermediate dose of 100 μM, amino phthalazinediones slow down electron flow, with concomitant effects on ATP production, DNA synthesis, and cell cycling, so that differentiation can proceed. At the high dose of 200 μM, amino phthalazinediones completely stop ATP production, DNA synthesis, and cell cycling in the stressed cell, such that the cell becomes quiescent but does not die.

Thus, phthalazinediones of the invention may be used to control cell fates and serve as redox buffers for the redox- and thiol-sensitive energy producing pathways in the mitochondrion, signaling pathways at the cell plasma membrane, and glutamate uptake and cytokine secretion by astrocytes in the central nervous system (Trotti et al., J. Biol. Chem. 271: 5976-5979, 1996). In particular, amino phthalazinediones catalyze disulfide cross-linkages in the adenine nucleotide translocase (ANT) of the mitochondrial anion channels and in the megapores, which prevents energy production, increases production of the potent signal transducers hydrogen peroxide (H2O2) and superoxide (O2-) (Zamzami et al., Oncogene 16: 1055-1063, 1998; Constantini et al., J. Biol. Chem. 271: 6746-6751, 1996), and liberates the apoptosis-inducing factors cytochrome c and AIF.

Under certain conditions, loss of redox control causes: (1) cross-linking of thiols in the adenine nucleotide translocase and other proteins, which then opens the mitochondrial transmembrane pores and channels and leads to a decline in mitochondrial voltage and energy production (Constantini et al., J. Biol. Chem. 271: 6746-6751, 1996; Larochette et al., Exp. Cell Res. 249: 413-421, 1999; Zanzami et al., Oncogene 16: 1055, 1998); (2) increases in intracellular calcium levels; (3) activation of redox defenses and heat shock proteins; (4) activation of redox-sensitive cell cycling factor AP-1 and E2F/Rb pathway; (5) activation of apoptotic pathways via AsK-1, with liberation of caspases, cytochrome c, and AIF from the failing mitochondrion; (6) a decline in ADP-dependent electron flow, as well as alteration of mobility of redox sensitive iron-sulfur proteins at mitochondrial Complex III (Zhang et al., J. Biol. Chem. 275: 7656-7662, 2000); (7) oxidation of macromolecules, including redox-sensitive proteins such as glutamate transporters (Trotti et al., J. Biol. Chem. 271: 5976-5979, 1996), mitochondrial DNA, and membrane lipids; (8) a failure in modulation of redox-sensitive phosphatases PTB-1, SHP-1, and SHP-2 (Doza et al., Oncogene 17: 19-26, 1998); and (9) dysregulation of the thiol-sensitive MAP kinase-Ras pathway, which controls cellular proliferation.

With redox support to buffer the redox stress and restore the redox status, the mitochondrion resumes energy production. The cell then repairs stress-induced damages caused by misfolded proteins, obesity-induced inflammation, heavy metal intoxication or Nrf2 transcription factor upregulation or downregulation, restocks essential substrates, and removes all offenders, in essence treating its own disease. To be successful, any exogenous redox agent must therefore enable the cell to correct the redox aberration, remove the cellular stress, and repair mechanical damages, without toxic side effects. Accordingly, in an embodiment of the invention, phthalazinediones primarily support metabolically distressed cells in a subject, by buffering the intracellular redox status without toxic side effects, to enable the subject's cellular repair or defense functions, rather than treat a particular condition in terms of trying to eliminate the disease or its cause.

Redox support therapy may be utilized in various disease states, such as in (1) inflammatory conditions where overactive cells, e.g., lymphocytes, macrophages, astrocytes, or microglia, strain redox defenses and energy production such as metabolic syndrome; (2) infectious conditions; (3) neurological disorders; (4) immune disorders; (5) proliferative diseases; (6) chelation therapy; and (7) senescence.

Metabolic Syndrome

Conditions of metabolic distress, includes redox imbalance or deficiency, metabolic syndrome (Syndrome X), intoxication, diabetes, insulin resistance, hyperglycemia, hypoglycemia, hyperinsulinemia, hypoinsulinemia, hypoadiponectinemia, hyper fatty acidemia, inflammation, tissue injury, and burns.

Inflammatory conditions where overactive cells, e.g., lymphocytes, macrophages, astrocytes, or microglia, strain redox defenses and energy production, include Parkinson's disease, Alzheimer's disease, Huntington's disease, multiple sclerosis (ms), Guillain-Barre syndrome (GBS, acute inflammatory demyelinating polyneuropathy, acute idiopathic polyradiculneuritis, acute idiopathic polyneuritis, or Landry's ascending paralysis), Lyme disease, Crohn's disease, ulcer, colitis, hemorrhoids, diarrhea, proctitis, arthritis, osteoarthritis, rheumatoid arthritis, stroke, myocardial infarction, auricular or atrial fibrillation, preexcitation syndrome (Wolff-Parkinson-White syndrome), arteriosclerosis, atherosclerosis, inflammation of blood vessels that characterize vascular disease in heart and brain, thromboangiitis obliterans (Winiwarter-Buerger disease), other inflammatory conditions of the vascular system, inflammatory conditions of the skin such as dermatitis, eczema, psoriasis, postoperative complications, peritonitis, bronchitis, and pleurisy.

Metabolic syndrome (MS) is a clustering of cardiovascular risk factors, with insulin resistance as a major feature. This syndrome generally consists of 3 or more of the following components: hyperglycemia, hypertension, hypertriglyceridemia, low HDL, and increased abdominal circumference and/or BMI at >30 kg/m². These components lead to obesity which in turn manifests into inflammation and insulin resistance. The manifested inflammation is one of the key processes underlying metabolic disease in obese individuals. Specifically, the adipose tissue of obese individuals express features that are characteristic of active local inflammation. This inflammation alters the functions of the adipose tissue thus leading to, for example, systemic insulin resistance. This obesity-induced inflammation also leads to coronary heart disease, insulin resistance and stroke. The present invention provides methods of treating or preventing the development of coronary heart disease (CHD), diabetes-2, stroke and other conditions related to obesity-induced inflammation by administering a redox support therapy, which includes a phthalazinedione composition, to a subject in need thereof. The redox support therapy modulates the obesity-induced inflammation thus preventing the further manifestations of a stroke, coronary heart disease or diabetes. These phthalazinediones, as described herein, are active agents used in the redox support therapy against the chronic inflammation that is frequently associated with MS. Inflammatory markers that have been associated with MS include hs-CRP, TNF-α, fibrinogen, and IL-6, among others. Cytokines are released into the circulation by adipose tissue, stimulating hepatic CRP production. The prothrombotic molecule PAI-1 is also increased in MS while adiponectin, produced exclusively by adipocytes, is decreased in obesity.

Adipocytes provide a flexible storage depot for excess nutrients, a property that creates a valuable resource during starvation. Adipocytes are also endocrine cells, secreting hormones that regulate energy intake and expenditure throughout the body. With overnutrition, however, adipocytes are pushed to the limits of their ability to store lipids and to regulate nutrient metabolism, and along with obesity comes an increase in the inflammatory marker expression. The cells of the innate immune system regulate these processes, in particular adipose tissue macrophages (ATMs), which make up a large proportion of the nonadipose cells in adipose tissue. ATMs infiltrate fat at the later stages of obesity and can cause some of the complications of the condition, particularly, insulin resistance.

In lean subjects adipose tissue macrophages (ATMs) have low inflammatory activity, restrained by T_{H}2 cytokines. However, in obese subjects, new macrophages are recruited to fat and stimulated by T_{H}1 signals, these macrophages' secretion of pro-inflammatory cytokines impair insulin signaling in adipocytes. This leads to increased lipolysis and the release of free fatty acids into the circulation. These fatty acids render the liver and skeletal muscle insulin resistant and contribute to a pre-diabetic state. This is an early and important event in the development of type-2 diabetes. In normalizing the inflammatory response, phthalazinediones, as used in the compositions and methods described herein, provide treatment and prevention of the development of type 2 diabetes. Obesity alters the properties of adipose tissue T cells before ATMs do and CD8+ adipose tissue T cells initiate the inflammatory cascade that leads to the insulin resistance in adipocytes. The redox support therapy provided by the phthalazinedione as used in the methods and compositions described or incorporated herein, are used to modulate the obesity induced inflammatory response. While not wishing to be bound by any specific theory, it is believed that these phthalazinediones modulate the obesity-induced inflammatory response by increasing the number of suppressor T cells. The modulation of the inflammatory response improves insulin sensitivity, prevents coronary heart attacks and strokes.

To survive any stress, cells must replace depleted thiols and maintain optimum mitochondrial redox potentials and activities. In one embodiment of the invention, therapy includes combined treatment with phthalazinediones and compounds to replace the lost thiols, oxidatively protect the subject, eliminate the source of stress, or otherwise support the subject in fighting a particular condition. A compound that is an amino acid, antibiotic, antiviral agent, anti-inflammatory agent, antioxidant, immunomodulator, reductant, oxidative protector, steroid, or vitamin is suitable. Compounds such as a cysteine (e.g., acetyl cysteine, N-acetylcysteineamide), glutathione, lipoic acid (e.g., alpha lipoic acid, dehydrolipoic acid), hydralazine, thioredoxin, biopterin (e.g., tetrahydropterin, sepiapterin), glucocorticoid, dexamethasone, rasagiline, ferulic acid, minocyline, menadione, tetracycline, isosorbate dinitrate, dextromethorphan, or mixtures thereof are also suitable. The additional compound may be administered simultaneously, separately, or sequentially. Preferably, the additional compound is administered simultaneously.

The preferred active ingredients may be formulated into a pharmaceutical composition with one or more pharmaceutically acceptable excipients. For example, a pharmaceutical composition may comprise a phthalazinedione, a glutathione, and one or more pharmaceutically acceptable excipients. The pharmaceutical composition may be in the form of a tablet, capsule, granule, powder, solution, suspension, microsphere, liposome, colloid, lyophilized composition, gel, lotion, ointment, cream, spray, or suppository and administered intravenously, intramuscularly, intraperitoneally, subcutaneously, orally, nasally, mucosally, transdermally, parenterally, vaginally, or rectally. A therapeutically effective amount of the phthalazinedione or a pharmaceutical composition comprising a therapeutically effective amount of the phthalazinedione is administered to a subject in metabolic distress, to maintain the desired redox status and mitochondrial energy production, as well as the redox-sensitive MAP kinase-Ras PT3K signal transduction pathways.

The amount of phthalazinedione needed or effective at any one point is cell- and stress-dependent. Optimum dosage and treatment require proper diagnosis of the thiol redox status of the patient's aerobic metabolism in the stressed mitochondria. Administration sufficiently early on in cell or stress development, such that cellular structures or functions have not deteriorated beyond repair, e.g., mitochondria swollen and leaky, cells entering apoptosis, would be particularly beneficial. The thiol redox status must also be frequently monitored, since phthalazinediones can be oxidatively very labile and rapidly expended.

In tissue culture, small doses of less than 1 μg/ml of an amino phthalazinedione are effective for conditions with chronic losses of cells, especially of stem or developing cells, as in neuroimmunodegenerative syndromes. In conditions where proliferation and apoptotic rates are out of control, including cancer, autoimmunity, infection, and traumas, doses greater than 50 μg/ml of amino phthalazinediones are required. Successful treatment with the phthalazinedione compounds of the invention therefore depends both on redox diagnosis with repeated assessment of cellular thiol redox status and on maintenance of proper dosage of the phthalazinedione over time. Treatment with phthalazinediones is directed at cells or organs in which stress has dysregulated thiol redox homeostasis, with resulting energy deprivation and oxidant stress.

In one embodiment of the invention, amino phthalazinediones also act as efficient substrates for reaction with many of the reactive oxygen species and radicals that are inevitably generated in the stressed mitochondrion. Because of their antioxidant, anti-inflammatory, antiproliferative, immunomodulatory, redox-buffering, and non-toxic properties, phthalazinediones are also beneficial as adjunctive support therapy for the stressed cell regardless of the compromising stress or its downstream symptoms. In rare disease states, redox support may be sufficient for the diseased cell to treat itself, but in some situations, the cell will also need the mechanical, pharmacological, or genetic support of standard medical treatments such as radiation, chemotherapy, laser therapy, surgery, medication, and nutrition used in treating particular disease conditions. As adjunct support therapy, the phthalazinediones of the invention may be administered simultaneously, separately, or sequentially for a combined treatment regimen. The following examples further illustrate the invention.

Heavy Metal Chelation

The chronic heavy metal load of the human body is increasing in today's world to the extent that, although not yet acutely toxic, it contributes to a decrease in the overall state of health and well-being of all of us. The present invention provides methods of effectively reducing the ion load of heavy metals such as iron, cadmium, lead, copper, mercury, aluminum, arsenic, nickel, and so on by modulating reactive oxygen species.

In certain embodiments of the invention, chelation therapies are provided wherein phthalazinediones, as described herein, act as efficient heavy metal chelators, alone or in combination with other agents.

The phthalazinediones described herein work more gently and are easier to administer than most commonly used chelators. During the treatment, a significant decrease in the body's heavy metals ions can be seen. This is accomplished without disturbing the mineral and trace element relationships. Unlike most other heavy metal detoxifications and chelating agents, the phthalazinediones do not cause loss of essential organic metals and minerals that are essential to your health. The phthalazinediones work to eliminate not just the unbound heavy metal ions but also the bound heavy metal ions.

Cadmium Chelation

For example, the monoisoamyl ester of 2,3-dimercaptosuccinic acid (hereinafter “DMSA”) containing two sulfhydryl groups is a potent chelating agent capable of mobilizing even intracellularly bounded cadmium. In the chelation therapy provided herein the phthalazinediones as described herein are administered in combination with monoisoamyl 2,3-dimercaptosuccinate (hereinafter “MiADMS”). The antioxidant effects of the phthalazinediones provide synergistic treatment of the cadmium intoxication by reducing the cadmium induced oxidative stress without affecting the chelating effects of MiADMS. The treatment of cadmium intoxicated animals with MiADMS reversed the cadmium induced increase in blood catalase, superoxide dismutase (SOD) and malondialdehyde (MDA), liver MDA and brain SOD and MDA levels. However, alone, MiADMS fails to modulate the decrease in blood, liver and brain of reduced glutathione (GSH) and the increase in oxidized glutathione (GSSG) levels caused by the cadmium intoxication. The administration of phthalazinediones reduced these cadmium induced alterations in the blood and liver GSH, GSSG, blood catalase, SOD, MDA and brain MDA levels without lowering blood and tissue cadmium contents. However, the combined treatment with MiADMS and phthalazinedione as described herein reversed these alterations as well as reduced blood and tissue cadmium concentrations. The combined treatment also improved liver and brain endogenous zinc levels, which were decreased due to cadmium toxicity.

Lead Chelation

The present invention also includes chelation therapy, with the phthalazinediones described herein, for the treatment of lead intoxication. Lead is a potent neurotoxicant that causes oxidative stress, which leads to numerous neurobehavioral and physiological alterations by binding to sulfhydryl groups or by competing with calcium. Lead causes a significant increase in reactive oxygen species, nitric oxide, and intracellular free calcium levels along with altered behavioral abnormalities in locomotor activity, exploratory behavior, learning, and memory. Lead not only targets the central nervous system (CNS) but it also haywires mitochondrial calcium homeostasis, intracellular oxidants levels, ATP production, and apoptogenic factors. This causes systemic mobilization and depletion of intrinsic antioxidants defense, membrane damages, destabilize calcium homeostasis and ultimately leads to apoptosis. Mitochondrial-dependent apoptosis are also results of lead intoxication.

In the present invention, phthalazinediones, as provided in the methods and compositions herein, are used in chelation therapy against lead intoxication. In combination with MiADMS, the phthalazinediones of the methods and compositions herein described, are administered to a subject in need thereof. MiADMS, as used herein, can be prepared by any known esterification process suitable for converting meso 2,3-dimercaptosuccinic acid (DMSA) to MiADMS. Lead-induced neurodegeneration is a further result of lead intoxication. Importantly, lead also induces oxidative stress via reactive oxygen species (ROS). Most of these alterations showed significant recovery following combined therapy with the phthalazinediones discussed herein and MiADMS. Specifically, many of the elevated brain oxidative stress variables were reversed by the coadministration of MiADMS and the phthalazinediones described herein. Chelation therapy with the phthalazinediones and MiADMS reduced lead-induced ROS and NO levels by 65-98.5% in both cases. More preferably, the level of ROS and NO reduction was between 70% and 98%. In certain other embodiments the ROS and NO levels were reduced to between 80% and 95%. In certain embodiments, the levels of ROS and NO was reduced by as much as 98.5%. The combination of MiADMS and the phthalazinediones described herein also recovered GSH, and SOD levels. In certain embodiments of the invention, combination chelation therapy with MiADMS and the phthalazinediones described herein recovered at least 80% in GSH. In certain embodiments the GSH recovery is at least 90% while in other embodiments the GSH recovery is at least 98%. SOD recovery was at least 50% in certain embodiments. However, the present invention also provides other embodiments wherein the SOD recovery is at least 65%. In other embodiments, the SOD recovery is at least 75%. The chelation therapy including phthalazinedione and MiADMS, in certain embodiments, reduced brain lead concentration by at least 70%, at least 75%, at least 80% or at leas 85%.

Lead intoxication was also treated with chelation therapy using MiADMS and the phthalazinediones herein described with the resulting effects that apoptosis in the brain was reduced. Cytochrome c release is a known marker for apoptotic cell death. Upon treatment with the combination chelation therapy of MiADMS and the phthalazinedione a decrease in the expression of cytochrome c is produced which indicates the reduction of apoptotic cell death in the brain mitochondria.

Learning and memory and other behavioral changes associated with lead intoxication are also improved by the combination chelation therapy of MiADMS and the phthalazinediones described herein. For example, the Norepinephrine (hereinafter “NE”), Dopamine (hereinafter “DA”) and Serotonin (hereinafter “5-HT”) levels are depleted by lead intoxication. However, combination chelation therapy, with DiADMS and phthalazinedione reversed the depleted biogenic amine levels towards normalcy. NE was reversed to about 85% and more preferably to at least 90%. DA levels were reversed to about 65% and more preferably to about at least 75% and more preferably to about 85%. 5-HT was reversed to at least about 85%. More preferable levels were obtained wherein the 5-HT levels were reversed to about at least 90% and even more preferably to at least about 95%.

DA controls arousal levels in many parts of the brain and is vital for giving physical motivation. Dopamine has many other functions in the brain, including important roles in behavior and cognition, voluntary movement, motivation and reward, inhibition of prolactin production, sleep, mood, attention, and learning. When levels are severely depleted—as in Parkinson's disease—people may find it impossible to move forward voluntarily. Low dopamine levels are also implicated in mental stasis. NE is mainly an excitatory chemical that induces physical and mental arousal and heightens mood. Norepinephrine plays a significant modulatory role in the acquisition of learning. NE is released during emotional arousal and plays a central role in the emotional regulation of memory. 5-HT transmission has been implicated in memory and in depression. Both 5-HT depletion and specific 5-HT agonists lower memory performance, while depression is also associated with memory deficits.

In lead-intoxicated subjects, lack of learning and memory is observed in comparison to normal subjects unaffected by lead intoxication. Treatment with the combination chelation therapy of MiADMS and phthalazinedione improved both learning and memory by reducing the lead concentration in the brain. In certain embodiments depletion of brain lead concentration was at least 65%. In other embodiments the depletion was at least 75%. In other specific embodiments the brain lead concentration was depleted at least about 85%.

Zinc and Copper Chelation

Both zinc and copper are essential minerals that are required for various cellular functions. However, these metals can be toxic in excess amounts. Zinc and copper homeostasis requires a coordinated regulation by different proteins involved in uptake, intracellular storage/trafficking and excretion of these metals. Zinc (hereinafter “Zn”) supports a healthy immune system, is needed for wound healing, and helps in maintaining sense of taste and smell. Zn is also essential for normal growth and development during pregnancy, childhood and adolescence. Similarly, copper (hereinafter “Cu”) is needed for formation of red blood cells, and keeps the blood vessels, nerves, immune system and bones healthy. Symptoms of Zn deficiency includes growth retardation, hair loss, diarrhea, delayed sexual maturation and impotence, eye and skin lesions, loss of appetite, weight loss, delayed healing of wounds, taste abnormalities and mental lethargy.

Along with the methods for treating cadmium and lead intoxication, the present invention also provides methods of modulating the effects of Zinc or Copper intoxication by chelation therapy. A chelation therapy is provided which combines phthalazinediones as described herein and calcium-ethylene-diamine-tetra-acetate (hereinafter “CaEDTA”). Other suitable combination chelation therapies include the phthalazinediones and N,N,N′, N′tetrakis (2-pyridylmethyl)ethylenediaminepentaethylen (hereinafter “TPEN”) or 1-hydroxypyridine-2-thione (hereinafter “pyrithione”). The combination therapy of the phthalazinedione and the pyrithione demonstrates the ability to attenuate neuronal death after Zn or Cu intoxication.

Amyloid beta (A-beta) is a defining feature of Alzheimer's disease. The neurotoxic and neuroprotective properties of A-beta are modulated by the binding to transitional metal ions such as Cu. Therefore, an interference with metal interaction can reverse the neurotoxic properties of A-beta. The phthalazinedione of the present invention provides the necessary interference to prevent and reverse the neurotoxic properties, because of its heavy metal chelating abilities.

In treating Zn or Cu intoxication, or any of the other metal intoxications discussed herein, the phthalazinedione that is coadministered also acts as an antioxidant to attenuate the oxidative damage caused by the heavy metal intoxication. In certain aspects of the invention disclosed herein, the phthalazinedione is coadministered, simultaneously, prior to or subsequent to the administration of a second agent. In certain aspects the second agent is desferrioxamine mesylate. These administrated compositions are herein defined as chelation therapy for iron overload or iron intoxication. The iron intoxication can result in acute iron overload disorders, such as, iron poisoning, hemocheomotosis and transfusional hemosiderosis resulting from frequent blood transfusions during the treatment of .beta.-thalassemia otherwise known as Cooley's Anemia, sickle cell anemia, aplastic anemia and some forms of leukemia.

Iron Chelation

In another aspect, the present invention also provides chelation therapy for modulating the effects of iron intoxication. The chelation therapy includes a phthalazinedione. Such phthalazinediones, bind tightly to iron in the body. While not wishing to be bound by any specific theory, it is believed that when the iron is bound to an iron chelator, the iron becomes inert and the reactivity of the iron is significantly dampened. In some embodiments, the phthalazinediones described herein, are co-administered with other chelation therapies. In certain embodiments of the invention, phthalazinediones are used in iron chelator therapy to prevent iron induced injury to cells. In certain other embodiments of the invention, phthalazinediones are administered to a subject to remove excess iron. While not wishing to be bound by any specific theory, it is believed that iron chelator therapy remove excess iron from the body so that the body's own repair mechanisms can be enabled to correct the damage caused by excess iron. In certain other embodiments, phthalazinediones are administered to a subject to neutralize free iron by blocking the ion's ability to catalyze redox reactions. In certain embodiments of this invention, conditions related to iron induced cell injuries are treated by methods of administering phthalazinedione to a subject in need thereof. In certain other embodiments, conditions related to free iron catalyzed redox reactions are treated by methods of administering phthalazinedione to a subject in need thereof. In certain preferred embodiments, the phthalazinedione is luminol. In certain other embodiments, the phthalazinedione is monosodium luminol.

The multi-organ oxidative stress in Fredrick's Ataxia (FA) may be due to loss of the highly redox-sensitive iron-containing enzyme, aconitase that is essential for electron transport and energy production in mitochondria. In the frataxin deficient mitochondria, aconitase loses its Fe/S complex with the result that the Krebs cycle stops and citric acid, iron and reactive oxygen species (ROS) accumulate in the now swollen dying mitochondria. Leakage of mitochondrial contents, primarily cytochrome c and iron, induce further oxidative stress with activation of apoptotic pathways and death of the FA neurons.

In certain embodiments of this invention, phthalazinediones are used as potent anti-inflammatory agents, especially in viral infection or autoimmune inflammation. In certain preferred embodiments the phthalazinedione is a luminol. In further preferred embodiments the phthalazinedione is a monosodium luminol. In certain embodiments, the phthalazinedione is administered to a subject such that FA is treated. Luminol, binds to and share electrons with redox active metals (copper and iron) or cysteines in proteins and in the presence of oxygen, generate H₂O₂, a major signaling molecule controlling life and death of cells. The loss of cytochrome c curtails productive electron transport and ATP production and induces the cytochrome c and caspase-dependent apoptotic pathways as well as the autophagy pathways.

Proteostasis Regulation

In certain embodiments of the invention, phthalazinedione act as proteostasis regulators. The term proteostasis regulator as used herein refers to therapies that are effective in treating genetic or degenerative disorders associated with deficiencies in protein homeostasis or the proteostasis network. Cellular proteins face constant challenges to their homeostasis or proteostasis. Defects in the proteostasis occurring as a result of ER and oxidative stress lead to many diseases including neurodegeneration and immunodeficiency.

The present invention also relates to methods for treating conditions characterized by dysfunction in protein homeostasis resulting in gain-of-function and/or loss-of-function diseases in patients in need thereof. The conditions encompass metabolic, oncologic, neurodegenerative and cardiovascular disorders. Loss-of-function diseases, e.g., lysosomal storage diseases (LSDs) including the neuropathic variety, cystic fibrosis, or al-antitrypsin deficiency-associated emphysema, are often caused by dysfunction in protein homeostasis, or proteostasis, sometimes resulting from mutations in proteins traversing the secretory pathway that compromise the normal balance between protein folding, trafficking and degradation. Gain of function disease often are age-onset related disease, e.g., amyotrophic lateral sclerosis, age-related macular degeneration, inclusion body myositosis, Alzheimer's disease, Huntington's disease or Parkinson's disease. As described herein, the innate cellular protein homeostasis machinery can be adapted to fold mutated enzymes that would otherwise misfold and be degraded, by administering to the cell proteostasis regulators e.g., small chemical compound proteostasis regulators, RNAi, shRNA, ribozymes, antisense RNA, or proteins, protein analogs or mimetics. The present invention provides methods for treating conditions characterized by dysfunction in protein homeostasis by administering proteostasis regulators which, by altering the composition of the proteostasis environment of the cytoplasm and/or the endoplasmic reticulum, can partially restore folding, trafficking and function to non-homologous mutant enzymes, each associated with a distinct lysosomal storage disease. The present invention also contemplates the coadministration of a chaperone with the proteostasis regulator. It may be possible to ameliorate loss-of-function and/or gain-of-function diseases by administering proteostasis regulators or administering a combination of a pharmacologic chaperone and a proteostasis regulator.

A method for treating a condition characterized by dysfunction in protein homeostasis in a patient in need thereof is provided which comprises administering to the patient a proteostasis regulator in an amount and dosing schedule effective to improve or restore protein homeostasis, and to reduce or eliminate the condition in the patient or to prevent its occurrence or recurrence. The condition can be a loss of function disorder, e.g., a lysosomal storage disease. The condition includes, but is not limited to, Gaucher's disease, α-mannosidosis, type IIIA mucopolysaccharidosis, Fabry disease, Tay-Sach's disease, Pompe disease, cystic fibrosis, and al-antitrypsin deficiency-associated emphysema. The proteostasis regulator functions to scavenge free radicals and thereby reducing the endoplasmic reticulum stress. In certain aspects the condition is a gain of function disorder, for example, a disorder causing disease such as inclusion body myositis, amyotrophic lateral sclerosis, age-related macular degeneration, Alzheimer's disease, Huntington's disease or Parkinson's disease. Treatment of a disease or condition with the proteostasis regulator can coordinately upregulate signaling via a heat shock response (HSR) pathway and/or an unfolded protein response (UPR) pathway, including upregulation of genes or gene products associated with these pathways. It is also clear that affecting signaling pathways associated with longevity and youthfulness is another approach to regulate the proteostasis network.

Proteostasis regulators provided herein, include phthalazinediones alone or in combination with other known proteostasis regulators, function by manipulating signaling pathways, including the heat shock response, the unfolded protein response, and longevity-associated signaling pathways, resulting in transcription and translation of proteostasis network components by removing free radicals such that ROS and NO production and/or accumulation are modulated.

A single proteostasis regulator should be able to restore proteostasis in multiple diseases, because the proteostasis network has evolved to support the folding and trafficking of many client proteins simultaneously. Proteostasis regulators influence the biology of folding, often by the coordinated increase in chaperone and folding enzyme levels and macromolecules that bind to partially folded conformational ensembles, thus enabling their progression to intermediates with more native structure and ultimately increasing the concentration of folded mutant protein for export. The phthalazinedione acts as a proteostasis regulator by scavaing the free radicals associated with the ROS produced, thus modulating the environment caused by the misfolded protein, such that the body's natural defense will correct the misfolding of the protein.

The invention is additionally directed to methods for treating conditions characterized by dysfunction in protein homeostasis by manipulating intracellular calcium homeostasis to improve defects in mutant enzyme homeostasis that lead to LSDs. It has been found that agents that reduce cytosolic calcium concentration and/or increase endoplasmic reticulum (ER) calcium concentration enhance the folding and activities of mutant enzymes associated with LSDs, such as Gaucher's disease, mannosidosis and mucopolysaccharidosis Type IIIA. Furthermore, increasing the calcium concentration in the ER enhances the activity of calcium-binding chaperone proteins. Therefore, one embodiment of the invention is directed to the treatment of an LSD by enhancing the folding of a mutant lysosomal enzyme by administering a phthalazinedione that increases the calcium concentration in the ER and/or decreases the calcium concentration in the cytosol and/or enhances the activity of calcium binding chaperones in the ER.

LSDs result from deficient lysosomal enzyme activity, thus the substrate of the mutant enzyme accumulates in the lysosome, leading to pathology. In many but not all LSDs, the clinically most important mutations compromise the cellular folding of the enzyme, subjecting it to endoplasmic reticulum-associated degradation instead of proper folding and lysosomal trafficking. A small molecule agent, such as a phthalazinedione, that causes the restoration partial mutant enzyme folding, trafficking and activity is desirable, particularly if a single agent could ameliorate multiple distinct lysosomal storage diseases by virtue of its mechanism of action. It is the reduction of oxidative stress activity by phthalazinedione that enhances the capacity of the endoplasmic reticulum to properly fold misfolding prone proteins.

In certain embodiments, proteostasis regulators, such as phthalazinedione, restore the natural balance of the proteostasis network. In other embodiments the proteostasis regulator folds mutated proteins. In certain other embodiments the proteostasis regulator controls biological pathways within the proteostasis network in treating or modulating the effects of degenerative disorders associated with protein build up, such as Huntington's disease and Alzheimer's disease. In certain embodiments of the present invention, genetic or degenerative disorders associated with deficiencies in protein homeostasis are treated by methods of administering phthalazinediones to a subject in need thereof. In certain other embodiments of the invention, the phthalazinedione is a luminol. In certain preferred embodiments, the phthalazinedione is monosodium luminol. Monosodium luminol promotes the normal processing of the mutant ts1 gPr80env in T cells, and it allows survival of these cells even though they are infected. While not wishing to be bound by one specific theory, it is believed that monosodium luminol affects this mechanism by interacting with the chaperone GRP78, in addition to its antioxidant effects.

It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Protein homeostasis” or “proteostasis” refers to controlling the concentration, conformation, binding interactions, e.g., quaternary structure, and location of individual proteins making up the proteome, by readapting the innate biology of the cell, often through transcriptional and translational changes by modulating the reactive oxygen species and scavenging the free radicals. Proteostasis is influenced by the chemistry of protein folding/misfolding and by numerous regulated networks of interacting and competing biological pathways that influence protein synthesis, folding, conformation, binding interactions, trafficking, disaggregation and degradation.

“Proteostasis regulators” include, among other things, small molecules, that enhance cellular protein homeostasis. Proteostasis regulators function by manipulating signaling pathways, including, but not limited to, the heat shock response or the unfolded protein response, or both, resulting in transcription and translation of proteostasis network components or by modulating the cellular environment such that the cellular system can be modulated by the body's natural defense mechanisms. For example, celastrol activates the heat shock response, leading to enhanced expression of chaperones, co-chaperones and the like. Proteostasis regulators often function by manipulating signaling pathways, including the heat shock response (HSR) pathway, the unfolded protein response (UPR) pathway, and Ca²⁺ signaling pathways that control longevity and protein homeostasis, and/or the transcription and translation of components of a given pathway(s) comprising the proteostasis network, including chaperones, folding enzymes, and small molecules made by metabolic pathways. Methods for treating a condition characterized by dysfunction in protein homeostasis in a patient in need thereof include both loss of function disease and gain of function disease associated with defective proteostasis, which can be remedied utilizing proteostasis regulators.

Intracellular regulatory signaling pathways that alter proteostasis include the “heat shock response (HSR)” which regulates cytoplasmic proteostasis, the “unfolded protein response (UPR)” which maintains exocytic pathway proteostasis and pathways associated with organismal longevity control that also control protein homeostasis. These include the insulin/insulin growth factor receptor signaling pathway and pathways associated with dietary restriction as well as processes associated with the mitochondrial electron transport chain process. Temporal cellular proteostasis adaptation is necessary, due to the presence of an ever-changing proteome during development and the presence of new proteins and the accumulation of misfolded proteins upon aging. Because the fidelity of the proteome is challenged during development and aging, and by exposure to pathogens that demand high protein folding and trafficking capacity, cells utilize stress sensors and inducible pathways to respond to a loss of proteostatic control. These include the “heat shock response (HSR)” that regulates cytoplasmic proteostasis, and the “unfolded protein response (UPR)” that helps maintain exocytic pathway proteostasis.

“Disaggregation pathway”, “disaggregation activity”, or “disaggregase” refers to an activity exhibited by many organisms including humans that disassembles or disassembles and proteolyzes protein aggregates, for example, amyloid proteins or their precursors.

“Aggregation pathway” or “aggregation activity” refers to an activity exhibited by an organism that assembles or aggregates a protein sometimes aggregating toxic precursors into less toxic aggregates. The integrity of protein folding could play a role in lifespan determination and the amelioration of aggregation-associated proteotoxicity.

“Unfolded protein response (UPR) pathway” refers to a stress sensing mechanism in the endoplasmic reticulum (ER) wherein the ER responds to the accumulation of unfolded proteins in its lumen by activating up to three integrated arms of intracellular signaling pathways, e.g., UPR-associated stress sensors, IREL ATF6, and PERK, collectively referred to as the unfolded protein response, that regulate the expression of numerous genes that function within the secretory pathway. Ron et al., Nat Rev Mol Cell Biol 8: 519-529, 2007; Schroeder et al., Ann Rev Biochem 74: 739-789, 2005. UPR associated chaperones include, but are not limited to BiP, GRP94, and calreticulin.

“Heat shock response (HSR) pathway” refers to enhanced expression of heat shock proteins (chaperone/cochaperone/folding enzymes) in the cytosol that can have an effect on proteostasis of proteins folded and trafficked within the secretory pathway as a soluble lumenal enzyme. Cytosolic factors including chaperones are likely essential for adapting the secretory pathway to be more folding and trafficking permissive. Bush et al., J Biol Chem 272: 9086-9092, 1997; Liao et al., J Cell Biochem 99: 1085-1095, 2006; Westerheide et al., J Biol Chem 279: 56053-56060, 2004.

HSR-associated chaperones include, but are not limited to Hsp/c40 family members, Hsp/c70 family members, Hsp/c90 family members, the Hsp/c 40/70/90 cochaperones including Aha1, auxilin, Bag1, CSP, as well as the small heat shock protein family members. The HSR pathway also directly influences the proteome residing and functioning in the cytoplasm.”

UPR-associated chaperones include, but are not limited to, GRP78/BiP, GRP94/gp96, GRP170/ORP150, GRP58/ERp57, PDI, ERp72, calnexin, calreticulin, EDEM, Herp and co-chaperones SIL1 and P581PK.

“Folding enzymes” refer to proteins that catalyze the slow steps in folding including, but not limited to, disulfide bond formation by protein disulfide isomerase (PDI) and peptidyl-prolyl cis-trans-amide bond isomerization by peptidyl prolyl cis-trans isomerase (PPI).

“Treating” or “treatment” includes the administration of the compositions, compounds or agents of aspects of the present invention to prevent or delay the onset of the symptoms, complications, or biochemical indicia of a disease, alleviating or ameliorating the symptoms or arresting or inhibiting further development of the disease, condition, or disorder (for example, a gain of function disorder or disease related to the accumulation of toxic aggregates, for example, Alzheimer's disease, Huntington's disease, age-related macular degeneration, inclusion body myositosis, and Parkinson's disease; or a loss of function disorder, for example, a lysosomal storage disease, cystic fibrosis, or al-antitrypsin deficiency-associated emphysema). “Treating” further refers to any indicia of success in the treatment or amelioration or prevention of the disease, condition, or disorder (e.g., a gain of function disorder or disease related to the accumulation of toxic protein aggregates or a loss of function disorder, e.g., a lysosomal storage disease), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of aspects of the present invention to prevent or delay, to alleviate, or to arrest or inhibit development of the symptoms or conditions associated with a gain of function disorder or disease related to the accumulation of toxic aggregates or a loss of function disorder, e.g., a lysosomal storage disease. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a gain of function disorder or disease related to the accumulation of toxic aggregates or a loss of function disorder, e.g., a lysosomal storage disease but does not yet experience or exhibit symptoms, inhibiting the symptoms of the disease (slowing or arresting its development), providing relief from the symptoms or side-effects of the disease (including palliative treatment), and relieving the symptoms of the degenerative disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition. The dosing schedule for administering proteostasis regulators to treat a particular disease or condition will likely be less frequent than the dosing schedule for other drugs used to treat the same disease or condition.

“Patient”, “subject”, “vertebrate” or “mammal” are used interchangeably and refer to mammals such as human patients and non-human primates, as well as experimental animals such as rabbits, rats, and mice, and other animals. Animals include all vertebrates and invertebrates, e.g., mammals and non-mammals, such as sheep, dogs, cows, chickens, Cenorhabditis elegans, Drosophila melanogaster, amphibians, and reptiles.

“Loss of function disease” refers to a group of diseases characterized by inefficient folding of a protein resulting in excessive degradation of the protein. Loss of function diseases include, for example, cystic fibrosis, lysosomal storage diseases, and Von Hippel-Lindau (VHL) Disease. In cystic fibrosis, the mutated or defective enzyme is the cystic fibrosis transmembrane conductance regulator (CFTR). One of the most common mutations of this protein is AF508 which is a deletion (A) of three nucleotides resulting in a loss of the amino acid phenylalanine (F) at the 508th (508) position on the protein. In one embodiment, the invention is directed to a method of treating a loss of function disease in a patient in need thereof comprising administering to said patient a proteostasis regulator in an amount effective to improve or restore activity of the mutated enzyme. In a further embodiment, the proteostasis regulator restores the activity of the mutated enzyme by promoting correct folding of the mutated enzyme.

“Lysosomal storage disease” refers to a group of diseases characterized by a specific lysosomal enzyme deficiency which may occur in a variety of tissues, resulting in the build up of molecules normally degraded by the deficient enzyme. The lysosomal enzyme deficiency can be in a lysosomal hydrolase or a protein involved in the lysosomal trafficking.

Gaucher's disease, first described by Phillipe C. E. Gaucher in 1882, is the oldest and most common lysosomal storage disease known. Type I is the most common among three recognized clinical types and follows a chronic course which does not involve the nervous system. Types 2 and 3 both have a CNS component, the former being an acute infantile form with death by age two and the latter a subacute juvenile form. The incidence of Type 1 Gaucher's disease is about one in 50,000 live births generally and about one in 400 live births among Ashkenazim. Kolodny et al., 1998, “Storage Diseases of the Reticuloendothelial System”, In: Nathan and Oski's Hematology of Infancy and Childhood, 5th ed., vol. 2, David G. Nathan and Stuart H. Orkin, Eds., W.B. Saunders Co., pages 1461-1507. Also known as glucosylceramide lipidosis, Gaucher's disease is caused by inactivation of the enzyme glucocerebrosidase and accumulation of glucocerebroside. Glucocerebrosidase normally catalyzes the hydrolysis of glucocerebroside to glucose and ceramide. In Gaucher's disease, glucocerebroside accumulates in tissue macrophages which become engorged and are typically found in liver, spleen and bone marrow and occasionally in lung, kidney and intestine. Secondary hematologic sequelae include severe anemia and thrombocytopenia in addition to the characteristic progressive hepatosplenomegaly and skeletal complications, including osteonecrosis and osteopenia with secondary pathological fractures. See, for example, U.S. Application No. 2007/0280925.

Fabry disease is an X-linked recessive LSD characterized by a deficiency of α-galactosidase A (α-Gal A), also known as ceramide trihexosidase, which leads to vascular and other disease manifestations via accumulation of glycosphingolipids with terminal α-galactosyl residues, such as globotriaosylceramide (GL-3). Desnick R J et al., The Metabolic and Molecular Bases of Inherited Disease 7: 2741-2784, 1995. Symptoms may include anhidrosis (absence of sweating), painful fingers, left ventricular hypertrophy, renal manifestations, and ischemic strokes. The severity of symptoms varies dramatically. Grewal, J. Neurol. 241: 153-156, 1994. A variant with manifestations limited to the heart is recognized, and its incidence may be more prevalent than once believed. Nakao, N. Engl. J. Med. 333: 288-293, 1995. Recognition of unusual variants can be delayed until quite late in life, although diagnosis in childhood is possible with clinical vigilance. Ko et al., Arch. Pathol. Lab. Med. 120: 86-89, 1996; Mendez et al., Dement. Geriatr. Cogn. Disord. 8: 252-257, 1997; Shelley et al., Pediatric Derm. 12: 215-219, 1995. The mean age of diagnosis of Fabry disease is 29 years.

Niemann-Pick disease, also known as sphingomyelin lipidosis, comprises a group of disorders characterized by foam cell infiltration of the reticuloendothelial system. Foam cells in Niemann-Pick become engorged with sphingomyelin and, to a lesser extent, other membrane lipids including cholesterol. Niemann-Pick is caused by inactivation of the enzyme sphingomyelinase in Types A and B disease, with 27-fold more residual enzyme activity in Type B. Kolodny et al., 1998, Id. The pathophysiology of major organ systems in Niemann-Pick can be briefly summarized as follows. The spleen is the most extensively involved organ of Type A and B patients. The lungs are involved to a variable extent, and lung pathology in Type B patients is the major cause of mortality due to chronic bronchopneumonia. Liver involvement is variable, but severely affected patients may have life-threatening cirrhosis, portal hypertension, and ascites. The involvement of the lymph nodes is variable depending on the severity of disease. Central nervous system (CNS) involvement differentiates the major types of Niemann-Pick. While most Type B patients do not experience CNS involvement, it is characteristic in Type A patients. The kidneys are only moderately involved in Niemann Pick disease.

Pompe disease (also known as glycogen storage disease type II, acid maltase deficiency and glycogenosis type II) is an autosomal recessive LSD characterized by a deficiency of α-glucosidase (also known as acid α-glucosidase and acid maltase). The enzyme α-glucosidase normally participates in the degradation of glycogen to glucose in lysosomes; it can also degrade maltose. Hirschhorn, The Metabolic and Molecular Bases of Inherited Disease 7: 2443-2464, 1995. The three recognized clinical forms of Pompe disease (infantile, juvenile and adult) are correlated with the level of residual α-glucosidase activity. Reuser et al., Muscle & Nerve Supplement 3: S61-S69, 1995.

Infantile Pompe disease (type I or A) is most common and most severe, characterized by failure to thrive, generalized hypotonia, cardiac hypertrophy, and cardiorespiratory failure within the second year of life. Juvenile Pompe disease (type II or B) is intermediate in severity and is characterized by a predominance of muscular symptoms without cardiomegaly. Juvenile Pompe individuals usually die before reaching 20 years of age due to respiratory failure. Adult Pompe disease (type III or C) often presents as a slowly progressive myopathy in the teenage years or as late as the sixth decade. Felice et al., Medicine 74: 131-135, 1995.

In Pompe, it has been shown that α-glucosidase is extensively modified post-translationally by glycosylation, phosphorylation, and proteolytic processing. Conversion of the 110 kilodalton (kDa) precursor to 76 and 70 kDa mature forms by proteolysis in the lysosome is required for optimum glycogen catalysis.

α-1 antitrypsin associated emphysema is one of the most common inherited diseases in the Caucasian population. The most common symptom is lung disease (emphysema). People with α-1 antitrypsin disease may also develop liver disease and/or liver cancer. The disease is caused by a deficiency in the protein alpha-1 antitrypsin. The development of lung disease is accelerated by harmful environmental exposures, such as smoking tobacco. α-1 antitrypsin disease has a genetic component. The age of onset, rate of progression, and type of symptoms vary both between and within families.

A “gain of function disease” refers to a disease characterized by increased aggregation-associated proteotoxicity. In these diseases, aggregation exceeds clearance inside and/or outside of the cell. Gain of function diseases are often associated with aging and are also referred to as “gain of toxic function” diseases. In one embodiment, the invention is directed to a method of treating a gain of function disease in a patient in need thereof comprising administering to said patient a proteostasis regulator in an amount effective to decrease aggregation of the protein. In a further embodiment, the proteostasis regulator decreases aggregation of the protein by promoting correct folding of the protein, inhibiting an aggregase pathway or stimulating the activity of a disaggregase. In a further embodiment, the proteostasis regulator would influence aggregation in a fashion that would decrease cytotoxicity.

Gain of function diseases include, but are not limited to neurodegenerative disease associated aggregation of polyglutamine repeats in proteins or repeats at other amino acids such as alanine. Lewy body diseases and other disorders associated with α-synuclein aggregation, amyotrophic lateral sclerosis, transthyretin-associated aggregation diseases, Alzheimer's disease, age-associated macular degeneration, inclusion body myositosis, and prion diseases. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: intracellular and extracellular aggregates of Aβ peptide and intracellular aggregates of the microtubule associated protein tau. Transthyretin-associated aggregation diseases include, for example, senile systemic amyloidoses, familial amyloidotic neuropathy, and familial amyloid cardiomyopathy. Lewy body diseases are characterized by an aggregation of α-synuclein protein and include, for example, Parkinson's disease. Prion diseases (also known as transmissible spongiform encephalopathies) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome. Fatal Familial Insomnia and Kuru.

Proteostasis regulators, such as the phthalazinediones described herein, can be used in a variety of methods for treatment of conditions characterized by dysfunction in protein homeostasis in a patient in need thereof. Thus, the present invention provides compositions and methods for treating diseases associated with a loss of function disorder, e.g., a lysosomal storage disease, or a gain of function disorder. In one embodiment, the composition includes small chemical compounds or biologics that act as a proteostasis regulator to upregulate signaling via a heat shock response (HSR) pathway, an unfolded protein response (UPR) pathway, and/or a Ca²⁺ signaling pathway, and a pharmaceutically acceptable carrier. In another embodiment, the composition comprises small chemical compounds or biologics that regulate protein chaperones by upregulating transcription or translation of the protein chaperone, or inhibiting degradation of the protein chaperone. In yet another aspect, the composition includes other small chemical compounds or biologics that upregulate an aggregation pathway or a disaggregase.

Molecular disorders of G proteins and signal transduction can result in gain of function disease or loss of function disease. Gain of function type diseases are caused by hyperactivity of Gα by suppression of GTPase activity. Mutations in as gene (gsp) and αi (gip2) generate endocrine tumors, and anomalous expression of gsp generates McCune-Albright syndrome and growth hormone-secreting pituitary adenoma. Gain-and-loss-of-function disease by AS mutation, i.e., Ala366 to Ser in as (αs-A366S) shows testotoxicosis and pseudohypoparathyroidism type Ia accompanying Albright hereditary osteodystrophy. The αs-A366S exhibits dominant-positive effects and dominant-negative effects. The αs-A366S mimics activation of Gs by the receptor, and exhibits temperature-sensitive features. Various modes of the loss-of-function of as have been identified and lead to a mechanism of the dominant-negative effects. Jikken Igaku 14(2): 219-224, 1996.

The proteostasis regulators described herein and the proteostasis regulators identified by the methods as described herein can be used in a variety of methods for treatment of conditions characterized by dysfunction in protein homeostasis in a patient in need thereof. Thus, the present invention provides compositions and methods for treating diseases associated with a loss of function disorder, e.g., a lysosomal storage disease, or a gain of function disorder. In one embodiment, the composition includes small chemical compounds or biologics that act as a proteostasis regulator to upregulate signaling via a heat shock response (HSR) pathway, an unfolded protein response (UPR) pathway, and/or a Ca²⁺ signaling pathway, and a pharmaceutically acceptable carrier. In another embodiment, the composition comprises small chemical compounds or biologics that regulate protein chaperones by upregulating transcription or translation of the protein chaperone, or inhibiting degradation of the protein chaperone. In yet another aspect, the composition includes small chemical compounds or biologics that upregulate an aggregation pathway or a disaggregase

The proteostasis regulator composition can be administered alone or in combination with other compositions. In one aspect, the proteostasis regulator is administered in combination with a pharmacologic chaperone/kinetic stabilizer specific to the disease or condition to be treated. In another aspect, the pharmacologic chaperone/kinetic stabilizer is one that is specific to the disease or condition to be treated. A pharmacologic chaperone/kinetic stabilizer that is specific to the disease or condition to be treated is a pharmacologic chaperone/kinetic stabilizer that stabilizes the folding of a protein associated with the disease or condition and/or associated with dysfunction in homeostasis. In a further aspect, the invention is a composition comprising a proteostasis regulator and a pharmacologic chaperone/kinetic stabilizer. In yet another aspect, the invention is directed to a method of treating a condition characterized by a dysfunction in protein homeostasis in a patient in need thereof comprising administering to the patient a proteostasis regulator in combination with a pharmacologic chaperone/kinetic stabilizer wherein said combination is administered in an amount sufficient to restore homeostasis of said protein.

The invention also encompasses a method of treating a condition characterized by a dysfunction in protein homeostasis in a patient in need thereof comprising administering to said patient a proteostasis regulator in an amount that restores homeostasis of the protein and does not increase susceptibility of the patient to viral infection. Also encompassed in the present invention is a method of treating a condition characterized by a dysfunction in protein homeostasis in a patient in need thereof comprising administering to said patient a proteostasis regulator in an amount that restores homeostasis of the protein and does not increase susceptibility of the patient to a tumor. In yet another embodiment, the proteostasis regulator does not enhance the folding of a viral protein or the synthesis of bacterial proteins. In a further embodiment, the proteostasis regulator does not enhance protein folding and trafficking capacity of tumor cells.

A proteostasis regulator composition, as described herein, can be used in methods for preventing or treating a method for treatment of a condition characterized by dysfunction in protein homeostasis in a patient in need thereof. The nature of the proteostasis regulator is of particular importance for the potential clinical usage as a factor to upregulate signaling via a heat shock response (HSR) pathway, an unfolded protein response (UPR) pathway, and/or a Ca²⁺ signaling pathway. The proteostasis regulator, e.g., a small chemical compound, thus has an unusual safety profile with minimum side effect as a survival molecule. It may therefore be used to treat a broad array of diseases related to a loss of function disorder, e.g., a lysosomal storage disease, or a gain of function disorder. The proteostasis regulator compositions therefore offers a new and better therapeutic option for the treatment of disease.

Preferably, treatment using proteostasis regulator compositions, in an aspect of the present invention, can be by administering an effective amount of the proteostasis regulator in an amount effective to improve or restore protein homeostasis in a patient in need thereof or to reduce or eliminate disease in the patient. As described above, a reduction in a disease encompasses a reduction or amelioration of one or more symptoms associated with the disease. Moreover, the proteostasis regulator compositions as provided herein can be used to reduce or eliminate a loss of function disorder, e.g., a lysosomal storage disease, or a gain of function disorder.

The invention is directed to methods of treating conditions associated with a dysfunction in protein homeostasis comprising administering to a patient a proteostasis regulator in an amount effective to improve or restore protein homeostasis. In one aspect of the invention, the condition associated with a dysfunction in the homeostasis of a protein selected from the group consisting of glucocerebrosidase, hexosamine A, cystic fibrosis transmembrane conductance regulator, aspartylglucsaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparan N-sulfatase, α-N-acetylglucosaminidase, α-glucosaminide N-acetyltransferase, N-acetylglucosamine-6-sulfate sulfatase, α-N-acetylgalactosaminidase, α-neuramidase, α-glucuronidase, β-hexosamine A and acid lipase, polyglutamine, α-synuclein, Ab peptide, tau protein and transthyretin.

A proteostasis regulator composition, useful in the present compositions and methods can be administered to a human patient per se, in the form of a stereoisomer, prodrug, pharmaceutically acceptable salt, hydrate, solvate, acid salt hydrate, N-oxide or isomorphic crystalline form thereof, or in the form of a pharmaceutical composition where the compound is mixed with suitable carriers or excipient(s) in a therapeutically effective amount, for example, to treat a proteostasis loss of function disorder, e.g., a lysosomal storage disease, or a gain of function disorder.

“Therapeutically effective amount” refers to that amount of the therapeutic agent, the proteostasis regulator composition, sufficient to result in the amelioration of one or more symptoms of a disorder, or prevent advancement of a disorder, cause regression of the disorder, or to enhance or improve the therapeutic effect(s) of another therapeutic agent. With respect to the treatment of a loss of function disorder, e.g., a lysosomal storage disease, or a gain of function disorder, a therapeutically effective amount refers to the amount of a therapeutic agent sufficient to reduce or eliminate the disease. Preferably, a therapeutically effective amount of a therapeutic agent reduces or eliminates the disease, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. “Therapeutic protocol” refers to a regimen for dosing and timing the administration of one or more therapeutic agents, such as a small chemical molecule composition acting as a proteostasis regulator.

Pharmaceutically acceptable carriers are determined in part by the particular composition being administered, as well as by the particular method used to administer the composition. Accordingly, there is a wide variety of suitable formulations of pharmaceutical compositions for administering the antibody compositions (see, e.g., latest edition of Remington's Pharmaceutical Sciences, Mack Publishing Co., Easton, Pa., incorporated herein by reference). The pharmaceutical compositions generally comprise a proteostasis regulator composition in a form suitable for administration to a patient. The pharmaceutical compositions are generally formulated as sterile, substantially isotonic and in full compliance with all Good Manufacturing Practice (GMP) regulations of the U.S. Food and Drug Administration.

Effective doses of the proteostasis regulator composition, for the treatment of a proteostasis loss of function disorder or gain of function disorder, as described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration of one or more proteostasis regulator compositions, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more proteostasis regulator polypeptides, or mimetic, analog or derivative thereof, with different binding specificities are administered simultaneously, in which case the dosage of each proteostasis regulator composition is usually administered on multiple occasions. Intervals between single dosages can be a few days, weekly, monthly or yearly. Intervals can also be irregular as indicated by measuring blood levels of the proteostasis regulator composition or the proteostasis network composition in the patient. In some methods, dosage is adjusted to achieve an concentration of 1-1000 μg/ml of proteostasis regulator composition and in some methods 25-300 μg/ml. Alternatively, the proteostasis regulator compositions can be administered as a sustained release formulation, in which case less frequent administration is required. Dosage and frequency vary depending on the half-life of the compound in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of a proteostasis loss of function disorder or gain of function disorder. Thereafter, the patent can be administered a prophylactic regime.

The phthalazinediones, as used in any of the methods and compositions described herein, can be administered by parenteral, topical, intravenous, oral, subcutaneous, intraarterial, intracranial, intraperitoneal, intranasal or intramuscular means for prophylactic as inhalants for proteostasis regulator compositions targeting a loss of function disorder, e.g., a lysosomal storage disease, or a gain of function disorder and/or therapeutic treatment. The most typical route of administration of an immunogenic agent is subcutaneous although other routes can be equally effective. The next most common route is intramuscular injection. This type of injection is most typically performed in the arm or leg muscles. Intramuscular injection or intravenous infusion are preferred for administration of antibody. In some methods, antibodies are administered as a sustained release composition or device, such as a Medipad™ device.

As to proteostasis regulators, the phthalazinediones described herein can optionally be administered in combination with other agents that are at least partly effective in treating a condition characterized by dysfunction in protein homeostasis in a patient in need thereof.

A proteostasis regulator composition for the treatment of a loss of function disorder, e.g., a lysosomal storage disease, or a gain of function disorder are often administered as pharmaceutical compositions comprising an active therapeutic agent, i.e., and a variety of other pharmaceutically acceptable components. See latest edition of Remington's Pharmaceutical Science (Mack Publishing Company, Easton, Pa.). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

For parenteral administration, compositions of aspects of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration is achieved by using the components as a mixture or as linked molecules obtained by chemical crosslinking or expression as a fusion protein.

In certain embodiments of the invention, the phthalazinedione acts as a modulator of nuclear factor erythroid 2-related factor 2 (Nrf2). Nrf2 is a transcription factor which regulates the expression of many detoxification and antioxidant enzymes. Nrf2 plays a significant role in adaptive responses to oxidative stress. Nrf2 belongs to the Cap N Collar (CNC-bZIP subfamily of basic/leucine zipper (bZIP) transcription factors. The Nrf2 transcription factor regulates expression of many detoxification or antioxidant enzymes. While not wishing to be bound by any specific theory, it is believed that the phthalazinedione modulates the Nrf2 transcription factor environment such that the activity of Nrf2 is increased and consequently its ability to translocate to the nucleus is improved. The Kelch-like-ECH-associated protein 1 (Keap-1) is a cytoplasmic repressor of Nrf2 that inhibits its ability to translocate to the nucleus. Nrf2 is a primary target of Keap-1 and Keap-1 interacts with Nrf2 and represses its function. These two proteins interact with each other through the double glycine-rich domains of Keap-1 and a hydrophilic region in the NEH2 domain of Nrf2. Keap-1 acts as a negative regulator of Nrf2 and as a sensor of xenobiotic and oxidative stresses. When cells are exposed to oxidative stress, electrophiles, or chemopreventive agents, Nrf2 escapes Keap-1-mediated repression and activates antioxidant responsive element (ARE)-dependent gene expression to maintain cellular redox homeostasis. Oxidative stress occurs in cells when the production of reactive oxygen species (ROS) exceeds antioxidant defenses. At low concentrations, ROS stimulates cell proliferation, but at higher concentrations, ROS damage cells by oxidizing proteins, DNA, and lipids, ultimately leading to cell death. Oxidative stress is also accompanied by depletion of reduced thiols, resulting in thiol deficiency in cells. Multiple cysteine residues allow Keap-1 to act as a molecular “switch” by responding to ROS with a conformational change, which releases Nrf2 to nuclear translocation, activating Phase 2 gene expression. When ROS levels increase in cells, the outward-facing cysteine residues in the Keap-1 molecule are oxidized, causing formation of disulfide bonds with other cysteines on the molecule, and altering the conformation of Keap-1 so that Nrf2 is released from it. Beyond its antioxidant function, Nrf2 is also a key factor regulating several genes that defend cells against the effects of environmental insults. Nrf2 activation confers protection against various pathologies, including cancer, neurodegenerative diseases, cardiovascular diseases, acute and chronic lung injury, autoimmune diseases, carcinogenesis, liver toxicity, respiratory distress and inflammation. The Nrf2-Keap-1 system is a major cellular defense mechanism against oxidative and xenobiotic stresses. Furthermore, the Nrf2-Keap-1 system contributes to protection against including and inflammation.

In certain embodiments of the present invention, phthalazinediones are administered to a subject such that the phthalazinedione buffers the interaction of Keap-1 and Nrf2. In certain other preferable embodiments, the phthalazinedione interacts with the glycine-rich portion of Keap-1 thus inhibiting its repressive effect on Nrf2. In certain embodiments of the present invention, conditions related to detoxification or antioxidant enzymes are treated by methods comprising administering a phthalazinedione to a subject in need thereof. In certain embodiments, the phthalazinedione is monosodium luminol. In certain embodiments, the phthalazinedione, or even more particularly, the monosodium luminol, decreases intracellular H₂O₂ levels in primary astrocyte cultures infected with ts1. Monosodium luminol reacts with free radicals such as ONOO⁻, prevents protein nitration and oxidation in cells and reduces markers of lipid peroxidation in CNS and thymus of ts1-infected mice. In certain embodiments, monosodium luminol is administered to a subject such that the amount of Nrf2 is stabilized such that phosphorylation and nuclear translocation is enhanced. Nrf2 is stabilized by monosodium luminol by inactivating the proteasomes that normally degrade Nrf2 and or by upregulating de novo synthesis of Nrf2 by transcriptional action. Monosodium luminol (GVT) reverses signs of oxidative damage without significant reduction of viral titer in the thymus and CNS of ts1-infected mice. Monosodium luminol protects against ts1-induced neurodegeneration, ND, by its antioxidant effects.

In certain other embodiments of the invention, phthalazinedione is administered to a subject such that protein conformational diseases are modulated or treated. Conformational diseases, or proteopathies, comprising clinically and pathologically diverse disorders in which specific proteins accumulate in cells or tissues of the body. The proteopathies include over 40 diseases, including Alzheimer's disease, Parkinson's disease, Huntington's disease, amyotrophic lateral sclerosis, prion diseases, inclusion body myopathy, and the systemic amyloidoses. Advancing age is an important risk factor for most proteopathies, in part because the ability to degrade or remove abnormal proteins becomes increasingly compromised in senescent cells. In certain embodiments of this invention, phthalazinediones are administered to a subject in need thereof, such that at least one of the diseases selected from Alzheimer's disease, Parkinson's disease, Huntington's disease, amyotrophic lateral sclerosis, prion diseases, inclusion body myopathy, or systemic amyloidoses is treated. In certain preferred embodiments, the phthalazinedione is luminol. Even more particularly, the phthalazinedione is monosodium luminol.

Shortly after generation, proteins normally fold into preferred, native conformations in which they can carry out their customary functions in the cell. In the proteopathies, a susceptible protein (usually one that is prone to misfolding and self-assembly) assumes an atypical, three-dimensional conformation, which often is enriched in β-structure. Proteins in this non-native conformation are highly stable, resistant to degradation, and have an enhanced tendency to aggregate with like protein molecules. These misfolded proteins can impart their anomalous properties to soluble, monomeric proteins having the same amino acid sequence. Hence, each proteopathy is characterized by a disease-specific buildup of aggregated proteins within cells or tissues, and the course of aggregation is sustained by the endogenous, seeded corruption and polymerization of newly generated proteins. Although an increase in β-structure usually precedes aggregation, in some instances proteins may first aggregate in their native-like conformation.

Some proteopathies can be induced by the introduction of pathogenic multimers of the aggregating protein. The best known such transmissible proteopathy is prion disease, which can be idiopathic, genetic, or infectious in origin. The prion diseases of humans include Creutzfeldt-Jakob disease, kuru, Gerstmann-Sträussler-Scheinker disease and fatal familial insomnia. In nonhuman species, they include scrapie, bovine spongiform encephalopathy (mad cow disease′), transmissible mink encephalopathy, chronic wasting disease of cervids, and others. Prion infectivity results from the corruption of endogenously generated (host) prion protein by exogenous, misfolded prions (proteinaceous infectious particles′). Both the structure of the infectious prions and the characteristics of the host govern transmissibility; molecular structural variations can influence the infectivity and pathogenicity of prions. Other proteopathies are generally considered to be non-transmissible, although some have been shown experimentally to be induced or accelerated by cognate proteinaceous seeds, including AA amyloidosis, systemic senile (apolipoprotein AII) amyloidosis, and Aβ-amyloidosis. In some instances, notably AA amyloidosis and ApoAII amyloidosis, protein deposition can be induced by heterogeneous molecular assemblies that happen to exhibit structural complementarity to the aggregating protein. The inducibility of proteopathy by corruptive templating indicates that a wide range of disorders may arise and propagate by similar molecular mechanisms. In certain embodiments of the present invention, a phthalazinedione is administered to a subject in need thereof, such that the protein aggregation is modulated or treated. In certain other embodiments the phthalazinedione is luminol or more preferably monosodium luminol. In certain embodiments the of the present invention the transmissible proteopathy is treated by the modulation of protein aggregation by administering phthalazinedione or more particularly luminol or even more particularly, monosodium luminol. In certain embodiments the proteopathy includes Creutzfeldt-Jakob disease, kuru, Gerstmann-Sträussler-Scheinker disease, fatal familial insomnia, scrapie, bovine spongiform encephalopathy (‘mad cow disease’), transmissible mink encephalopathy, chronic wasting disease of cervids, and others.

The endoplastic reticulum (ER) is a group of channels where the unfinished protein products of ribosomes are translated into active proteins and marked for distribution to various cellular compartments. These protein translation and transport processes in ER require large amounts of energy (ATP) from the nearby mitochondria and also require careful maintenance of their internal redox and anionic status. This process in ER also requires storage of large amounts of calcium for signaling when stressed. In addition multiple chaperones to assist in protein folding and transport through the ER are required. With cyclic deprivation of electron donors and acceptors, glucose and oxygen, both ER and mitochondria try to adapt to this metabolic redox stress, but often fail. This failure is accentuated by agents that specifically clog protein translation and transport in ER or block calcium uptake in ER and is ameliorated by redox-active agents that either block reactive oxygen species (ROS) production by cytochrome oxidase in mitochondria or scavenge ROS. Treatment with various ROS scavengers and antioxidants ameliorate the oxidative damages by scavenging ROS and/or by up-regulating various survival factors, including PERK-Nrf2, PKB/Akt, Bc12, protein phosphatase 2A and various ER chaperones. In certain embodiments of the present invention, methods are disclosed wherein a phthalazinedione is administered to a subject such that the phthalazinedione ameliorates oxidative damage by scavenging ROS or by upregulating the various survival factors. In certain embodiments the phthalazinedione is luminol. In further embodiments the phthalazinedione is a sodium luminol.

Many age-related neuro-muscular degenerative diseases, including IBM, Huntington's, SCA-3, Parkinsonism, Alzheimer's disease and others, are associated with accumulation of misfolded, aggregated, oxidized, mutated proteins or proteins containing large numbers of amino acid repeats with aberrant acetylation/deacetylations. A primary cause of the oxidant stress and cell death in IBM, and likely in other proteinopathies, is the faulty transport and maturation of proteins in the protein-clogged stressed ER with release of calcium phosphate from protein-clogged ER. Such constant calcium signaling and acidity and energy-requiring uptake of calcium by the now overworked electron transport in mitochondria further increases production of ROS. Proper treatment for these ER-induced proteinopathies is, of course, removal or digestion of the offending protein ‘clogs’. In certain embodiments of this invention, treatment with redox-active buffers such as phthalazinediones facilitates clearance of the aggregates. With the oxidant stress the clogging peptides in ER become oxidized and nitrated, treatment with redox-active buffers, such as luminol, maintains the peptides in a more reduced state. In certain embodiments, the phthalazinedione is monosodium luminol.

It should be understood that the present invention includes the treatment of all of the diseases and or conditions described herein by the administration of a phthalazinedione. Even more particularly, it should be understand that all the diseases and conditions herein described are treated with luminol and even more particularly monosodium luminol. The treatment can be by some of the mechanisms described herein or can by any mechanism that leads to the treatment of the disease or condition.

A proteostasis regulator composition for the treatment of a loss of function disorder, e.g., a lysosomal storage disease, or a gain of function disorder are often administered as pharmaceutical compositions comprising an active therapeutic agent, i.e., and a variety of other pharmaceutically acceptable components. See latest edition of Remington's Pharmaceutical Science (Mack Publishing Company, Easton, Pa.). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of aspects of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

### EXAMPLES

The following examples serve as exemplary embodiments or demonstrations of the inventions herein described and are should not be construed as being exhaustive of all possible embodiments or demonstrations.

### Example 1: Uncontrolled Inflammation

In inflammatory conditions, such as acute infections, wounds, and immune responses, phthalazinediones, especially amino phthalazinediones, quickly ameliorate the painful redox-induced edematous swelling and facilitate rapid healing. Edematous inflammatory lesions in intestines, such as duodenal ulcers, ulcerative colitis, and acute vascular injury, are all suppressed to some degree by thiol redox modulators, including dihydrolipoates, reduced biopterins, amino phthalazinediones, and more slowly by glucocorticoids. Healing rates increase, with replacement of the injured epithelial cells by thiol redox-stimulated new cell growth. Thus, phthalazinediones, acting as thiol redox modulators, suppress injurious over-reactive inflammatory responses and also facilitate healing and replacement of injured cells.

### Example 2: Uncontrolled Proteolysis

In conditions with aberrant or uncontrolled proteolysis, as in apoptosis or necrosis, thiol redox modulators, especially thioredoxin, either upregulate or downregulate the regulatory proteases involved in processing and digesting the thiol redox dependent caspases, endonucleases, and histone deacetylases responsible for protein and DNA hydrolysis. Diamide, a phthalazinedione with activity similar to the oxidized 4-amino phthalazinedione, activates and cross-link proteases that hydrolyze procaspase 3 to the active caspase fragments that, along with cytochrome c, initiate the apoptotic cascade in the nucleus.

Since these cross-linking agents also oxidize essential membrane proteins, such as the adenine nucleotide translocase in mitochondria or amyloid protein fragments in brain, the result is membrane pore formation in mitochondria with increased reactive oxygen species and cell destruction (Ueda et al., J. Immunol. 161: 6689-6695, 1998). Thus, reduced phthalazinediones will up- or down-regulate redox-sensitive proteases and thereby dictate life and death of stressed proliferating cells.

### Example 3: p53 and Aging

In conditions where cell growth and tumor formation are constantly suppressed by growth suppressor genes like p53, signs of premature aging and replication senescence appear early (Tyner et al., Nature 415: 45-50, 2002). Chronic cell losses in skin, hair, bone, adipose tissue, and the immune system occur. The p53 protein is a potent transcription factor that suppresses cell growth and DNA synthesis and is also an activator of genes that induce oxidative stress and apoptosis, such as Bax and caspases 3 and 9.

Thiol redox modulators such as phthalazinediones, which maintain cellular replication pathways by modulating cellular redox status, will override the p53-induced suppression and maintain a balance between apoptotic or proliferation pathways, depending on dosage. Since thiol redox modulators beneficially balance rates of cell death and proliferation in other syndromes of premature aging, including XPD deficiency and retrovirus-induced degenerative diseases, phthalazinediones acting as that thiol redox modulators, at appropriate dosages, and will re-balance the p53-induced thiol redox potential and thereby prevent the degenerative sequelae.

In other neurodegenerative syndromes in which aberrant peptides accumulate, including Alzheimer's and Parkinson's diseases, presenilin or synucleins may be responsible for accumulation of the Lewy bodies and β-amyloid peptides. Accumulation of these hydrophobic peptides in plasma, mitochondrial, or endoplasmic reticulum membranes of the cell may be responsible for the neuronal losses in these syndromes. These toxic peptides, like the polyglutamine proteins in Huntington's disease, also lead to astroglia-induced imbalances in thiol redox metabolism, with cell swelling, membrane leakiness, and mitochondrial necrosis. Maintenance of thiol redox status with reduced thiol redox modulators, especially an amino phthalazinedione and acetyl cysteine, will prevent or delay the neuronal death in these degenerative diseases (Wolfe and Selkoe, Science 296: 2156-2157, 2002; Welhofen et al., Science 296: 2215-2218, 2002).

### Example 4: NMDA-Induced Excitotoxicity Model

In NMDA-induced neuronal excitotoxicity, secreted microglial inflammatory products—glutamate, quinolinic acid, inflammatory cytokines, tumor necrosis factor, IL-1B, superoxide (O₂⁻), and nitric oxide (NO)—are likely responsible for the neuronal necrosis (Tikka and Kolstinaho, J. Immunol. 166: 7527-7533, 2001). These excitotoxins all rapidly perturb redox homeostasis in neurons, which slowly die, and in activated astroglia, which become activated and proliferate.

Minocycline, a cyclic polyhydroxy ketonic amide, which suppresses mitochondrial activity, prevents both the NMDA-induced proliferation of and toxic secretions by activated astrocytes, as well as the subsequent neuronal death (Tikka and Kolstinaho, J. Immunol. 166: 7527-7533, 2001). Cell death in neurons, secretory proliferative activation of astroglia, and proliferative response in astrocytes in the spinal cord are mitochondrial redox-mediated and correction of thiol redox status by phthalazinediones leads to control the fate of these brain cells.

### Example 5: Oxygen-Based Model

In acute metabolic distress, as in hypoxia, redox-sensitive transcription factors such as H1FA are rapidly activated, or under-activated if the oxygen deprivation is not too severe. These transcription factors are triggered by the alternate redox-sensitive mammalian target of rapamycin (mTOR) signal transduction pathway, which is upregulated by low oxygen, ATP, and amino acids. Activated mTOR markedly upregulates DNA synthesis and cellular proliferation, especially in endothelial and vascular smooth muscle cells. Consequently, mTOR is involved in many redox-sensitive proliferative diseases of vascular tissues, including diabetic retinopathy, psoriasis, rheumatoid arthritis, certain tumors, and arteriosclerosis (Humar, FASEB J. 16: 771-780, 2002).

Whether mTOR or its upstream activators are redox sensitive is not clear. Nonetheless, oxygen at low dose, like amino phthalazinediones at low dose, will increase proliferation, whereas oxygen at very low dose (<1%), or phthalazines at high dose, will stop proliferation and will activate cell death pathways. Vascular cell fates are clearly dependent on external redox agents that modulate internal redox status, and the responses and fates of these cells are readily controlled in a dose-dependent manner by external redox agents such as oxygen, amino phthalazinedione, diamide, or permeant thiols, which modulate the mTOR-signaling pathway. These redox agents are therefore useful as redox buffers in controlling the redox-sensitive mTOR pathway, ameliorating various vascular proliferative inflammatory diseases, and controlling angiogenesis both in tumor growth and inflammatory syndromes, particularly in brain.

### Example 6: Uncontrolled Oxygen Models

In uncontrolled oxygen metabolism, oxygen is not fully reduced, such that reactive oxygen intermediates accumulate. Cell fate is highly dependent on the concentration, location, and longevity of reactive oxygen species such as O₂⁻, H₂O₂, OH., NO, and OHOO. In proliferating vascular smooth muscle cells, addition of O₂ or H₂O₂ quickly increases DNA synthesis, via activation of the Id3/E2F pathway. In the presence of iron plus H₂O₂, which produces the more potent OH. radical, DNA synthesis, Id3 protein, and Id3 mRNA rapidly decline, while cell death rates increase. Thus, the fate of growing smooth muscle cells is highly dependent on oxygen redox status.

The two oxygen redox-sensitive genes, Id3 and GKLF, which are differentially responsive to oxygen redox status, are most sensitive to rapid changes in concentrations of reactive oxygen species. With increased concentrations in OH., Id3 expression is downregulated, GKLF expression is upregulated, and DNA synthesis ceases (Nickenig et al., FASEB J. 16: 1077-1086, 2002). The GKLF protein, when oxidized, is activated and inhibits Id3 expression by binding to the Id3 promoter. The Id3 protein, when reduced, is activated and upregulates the E2F-controlled proliferation pathway.

Thus, oxygen redox status, like thiol redox status, is a potent regulator of cell fates. Moreover, the two redox pathways, and the two electron acceptors oxygen and sulfur, interact repeatedly. For example, reduced phthalazines or thiols chemically reduce most of the reactive oxygen species, including peroxynitrite (ONOO⁻). Tetrahydropterin (BH4), a major cellular reductant in the central nervous system, reduces reactive oxygen species and the inducible oxidase iNOS. Under redox stress, in the presence of tetrahydropterin, iNOS produces nitric oxide (NO). Under redox stress when tetrahydropterin or reduced thiols are limited, iNOS produces superoxide (O₂⁻). In turn, superoxide (O₂⁻) or hydrogen peroxide (H₂O₂) activates Id3 and the E2F-controlled DNA synthesis pathway but only in the absence of iron or copper (Dehmer et al., J. Neurochem. 74: 2213-2216, 2000; Husman et al., FASEB J. 10: 1135-1141, 2002; Liberatore et al., Nature Med. 5: 1403-1409, 1999).

Thus, intracellular redox homeostasis, whether oxygen or thiol-mediated, is dependent on concentrations of cellular reductants—tetrahydropterin, glutathione, cysteine, NADPH—and cellular oxidants—O₂⁻, H₂O₂, NO, OH., Fe³⁺—as well as on concentrations of permeant extracellular reductants—reduced thiols, tetracyclines, phthalazines—and permeant extracellular oxidants-O₂⁻, gamma radiation, doxorubicin, glucocorticoids, cis-platinum, doxirubicin, etc. Consequently, redox homeostasis can be readily maintained by appropriate doses of permeant redox agents, notably by phthalazinediones, and with protean therapeutic implications. Phthalazines, tetracyclines, or thiols (Tikka and Kolstinaho, J. Immunol. 166: 7527-7533, 2001) potentially dictate and control the cell fate in activated or stressed cells, whether the disease-inducing redox imbalance is oxygen- or sulfur-mediated. In addition to controlling proliferation and activation pathways, these redox modulators also scavenge destructive oxygen radicals and thereby prevent apoptotic and necrotic pathways.

Potential therapeutic usefulness of these redox modulators in astroglia induced neurodegenerative diseases (Tikka and Kolstinaho, J. Immunol. 166: 7527-7533, 2001), in renal allografts (Husman et al., FASEB J. 10: 1135-1141, 2002), and in inflammation-induced cell damages (Ryan et al., Curr. Opinion in Rheumatology 8: 238-247, 1996) are now being recognized. Thus, redox modulating compounds, especially phthalazinediones, that modulate both the oxygen and sulfur redox pathways are proving to be therapeutically useful in situations where the patient's redox mechanisms are out of control.

### Example 7: Chronic Inflammation Model with Accumulation of Excess Lipids

In situations where foreign fats such as oxidized fatty acids or cholesterol accumulate, a chronic inflammatory reaction ensues. Signaling and transport processes in lipid-laden membranes falter. Lipid-laden activated macrophages accumulate. Oxidant stress follows, due to deficiency in glucose transport in the lipid-laden membranes and the increased production of oxidants and proteases by the influx of activated macrophages. Chronic localized abscesses form. In vascular tissue, atherosclerosis with occlusive diseases, stroke, myocardial infarction, cystic mastitis, wet macular degeneration, and engorged activated adipocytes are the result. In all these syndromes, thiol redox homeostasis becomes gravely perturbed and cellular redox damage occurs. Metabolic syndrome, or Syndrome X, with insulin resistance is an early sequela.

Therapies known to modulate the above lipid- and redox-induced syndromes include: (1) thiol redox modulators, especially amino phthalazinediones, to buffer the aberrant thiol redox status; (2) anti-proteases, especially minocycline, to block the excess proteolytic activity and suppress O₂⁻ production by the induced NO synthase by macrophages; (3) peroxisome proliferators, to accelerate oxidation of accumulating lipids; (4) caloric restriction, to block input and accumulation of the aberrant lipids and O₂⁻; (5) glucocorticoids, to deplete thiols by excretion, inhibit growth, and accelerate death of the overactivated macrophages and microglia; and (6) sepiapterin, to prevent superoxide (O₂—) production by iNOS in the brain and to prevent activation of the apoptosis stimulating kinase AsK-1, especially in the brain.

Many external therapies are therefore available to modulate and prevent the chronic abscess formations induced by accumulation of aberrant oxidized fats in cell membranes. To fully maintain optimum redox status, over time, in disease states with differing etiologies, various combinations and doses of all six redox approaches may be required. With optimum redox support, the subject will repair most damages and induce the means—for example, peroxisome proliferator receptors (PPARs) and adiponecting—to remove the offending fats. In severe defects, specific anti-proteases and antioxidants as those listed above are essential for optimal therapy. The phthalazinediones of the present invention will provide the redox support therapy for modulating the inflammatory response.

### Example 8: Redox-Controlled Neuronal Survival

Oxidizing agents such as H2O2, NMDA agonists, and N-nitrosoguanidines rapidly kill primary neurons. In the presence of oxidants the redox-sensitive nuclear poly (ADP-ribose) polymerase, which cleaves NAD+ to ADP-ribose and stabilizes nuclear proteins by ADP-ribosylating them, is rapidly activated. This depletes the neuron of NAD+ as well as the reductants NADH and NADPH. This also rapidly facilitates nuclear uptake of the mitochondrial redox-sensitive flavoprotein, apoptosis inducing factor (AIF).

These oxidants also open the redox-sensitive permeability transition pores and anion channels in mitochondrial membranes, which release AIF. AIF is then taken up by the poly (ADP-ribose) polymerase-activated nucleus to initiate chromatin condensation. Chromatin condenses, mitochondria grow swollen, and mitochondrial processes become uncoupled. Mitochondria then produce more oxidants, O₂⁻ and H₂O₂, and produce less ATP. In addition, the oxidants rapidly induce reshuffling of plasma membrane ionic phospholipids with surface exposure of phosphatidyl serine. This rapidly alters permeability and transport activities in plasma, mitochondrial, endoplasmic reticulum, and nuclear membranes. The plasma and endoplasmic reticulum membranes leak calcium, which activates innumerable signal transduction pathways, including ATM, mTOR, and p38 MAPK (Yu et al., Science 297: 259-263, 2002; De Giorgi et al., FASEB J. 10: 607-609, 2002).

Thus, redox status of most cellular membranes is rapidly altered by brief exposure to permeant oxidants, and cell death rapidly ensues, through both apoptotic (nuclear) and necrotic (plasma membrane) changes. Membrane-permeant reductants, such as phthalazinediones plus reduced biopterins and thiols, are able to buffer and maintain the proper redox status in membranes of oxidant-stressed organelles as occurs in acute neurodegenerative syndromes such as hypoxia or glucose deficient states, or in chronic inflammatory states such as Parkinson's disease, Alzheimer's disease, ALS, MS, AT, or aging.

### Example 9: Role of Thiol Redox Status in Mitochondrial Activities

The major source of chemical energy and heat in aerobic cells is mitochondria. The modulatable permeable pores and channels in mitochondria are exquisitely sensitive to thiol redox status. The specific mitochondrial channel is composed of two thiol redox sensitive proteins located in the inner membrane—adenine nucleotide translocase (ANT) and voltage dependent anion channel (VDAC)—and other coproteins such as cyclophilin D, hexokinase, benzodiazepine receptors, and the Bc1-2/Bax family of peptides. These proteins together control the permeability and transport of mitochondrial transmembrane channels and pores, which control ADP entry, proton exit, electron flow, intracellular calcium concentration, and O₂-production.

Bax, benzodiazepine receptors, and hexokinases, which bind to the outer membrane of mitochondria, regulate transport and pore formation in these membranes. Major physiologic modulators of this mitochondrial transmembrane pore include: (1) transmembrane voltage, which is generated by electron and proton gradients; (2) inducible membrane proteins, Bc1-2 and Bax; and (3) thiol redox status, the redox state of Cys-56 on the channel protein ANT being a major regulator of the permeability of mitochondrial transmembrane pores (He and Lemasters, FEBS Letters 512: 1-7, 2002).

Thiol oxidants or cross-linking agents such as diamide or diethyl maleate distort and open mitochondrial pores and channels, and uncouple electron flow, allowing oxygen to trap electrons and produce O₂—, H₂O₂, and other radicals. Energy production declines, and mitochondria release cytochrome c, caspases, and AIF. Destructive cytochrome c, redox-sensitive proteases, and caspases are activated in the cytoplasm and the nucleus, causing cell death, both apoptotic and necrotic.

Reduced thiols, dithiothreatol, glutathione, N-acetyl cysteine, or agents such as Bc1-2, Bongkrekic acid, cyclosporine A, or chaperone cyclophilins that can stabilize ANT sulfhydryls and maintain pore permeability status can completely prevent the electron leak and the cell death (Armstrong and Jones, FASEB J. 16: [online], Jun. 7, 2002; Castantini et al., Oncogene 19: 307-314, 2000; Hong et al., FASEB J. 16: 1633-1636, 2002).

Under oxidant stressed conditions, including radiation, chemotherapy, occlusive vascular diseases, leptin-deficient or resistin-induced obesity, caloric excesses, and type II diabetes, in which optimal thiol redox status is not maintained by the diseased adipose tissue of the patient, therapeutic support by external thiol redox buffers will be acutely necessary, at least until the patient can repair and buffer the stressed and imbalanced thiol redox status and fully activate its hypoxia-inducible transcription factors (Wenger, FASEB J. 16: 1151-1162, 2002; De Giorgi et al., FASEB J. 10: 607-609, 2002).

Depending on the type of oxidative stress, labile vicinal cysteinyl residues on ANT undergo cyclic oxidation, ionization, and eventually cross-linking. These oxidations and cross-linkages of protein thiols greatly perturb channel functions, especially by thiol cross-linking cyclic amines, diazenes (diamide), or phenylarsines. Uptake of ADP fails, protons are released with collapse of the inner membrane potential, ordered electron flow at mitochondrial Complex III falters, and O₂ now accepts the fluxing electrons with production of O₂— and other radicals. The oxidant-producing mitochondria release cytochrome c and AIF, and downstream oxidation of NFKB, AP1 (major transcription factor for proliferation), AsK-1 (apoptosis stimulating kinase), glutathione, Bax, HDAC (histone deacetylase in nucleus), PTEN (phosphatase in cytoplasm), and ATM occurs. Apoptosis, senescence, quiescence, or necrosis results, depending largely on the extent and duration of the redox stress.

A photoactive diamine fluorescent cation, tetramethyl-rhodamine, which accumulates in mitochondria and releases free radicals when photoactivated, is a potent agonist of the mitochondrial transmembrane pore. When tetramethyl-rhodamine is activated, all downstream effects of oxidation and cross-linking of ANT's labile cysteinyl residues occur, including translocation and polymerization of Bax in mitochondrial membranes. These effects are fully inhibited by Bongkrekic acid, a specific inhibitor of mitochondrial transmembrane pores (De Giorgi et al., FASEB J. 10: 607-609, 2002), as well as by reduced thiols, reduced phthalazines, cyclophilins, and pterines. Accordingly, the fate of cells under stress is largely dictated by mitochondrial thiol redox status, and that cell fates are readily buffered or controlled by permeant lipophilic redox-sensitive amines, such as phthalazinediones, tetrahydrobiopterin, and permeant thiols.

### Example 10: Thiol Redox Status in Mitochondria in Cancer Treatment

Controlling entry and exit of small molecules—Ca2+, H+, O2—and substrate anions through the redox- and voltage-sensitive mitochondrial channels and pores is to control cell fates. These channels and pores modulate concentrations of intracellular cations Ca2+ and H+, intracellular anions ADP, ATP, malate, and glutamate, and intracellular thiols, glutathione, cysteine, thioredoxin, and biopterin. By these means, these channels can indirectly modulate redox-sensitive sites in signal transduction, proliferation, development, transcription, apoptosis pathways, and necrosis pathways, thereby dictating cell fates.

Many agents that can directly modulate these pores are in use for antiproliferative therapies, notably as treatments for hyperproliferative syndromes and cancer (Miccoli et al., J. Nat. Cancer Inst. 90: 1401-1406, 1998; Ravagnan et al., Oncogene 18:2537-2546, 1999; Larochette et al., Exp. Cell Res. 249: 413-471, 1999). Three broad classes of modulators are in use—lipophilic peptides, lipophilic amines, and thiol redox-reactive cyclic amines.

Lipophilic peptides are useful as antiproliferative and anti-inflammatory therapies. These peptides, primarily Bax, Bc1-2, and cyclosporine A, either block or bypass mitochondrial transmembrane channels by creating pores of oxidized polymerized peptides of variable permeability in the mitochondrial outer membranes (De Giorgi et al., FASEB J. 10: 607-609, 2002). The redox-insensitive lipophilic benzo amines are useful in cancer therapy. Diazepam and lonidamine, for example, bind to mitochondrial benzodiazepine receptors in the mitochondrial matrix, block mitochondrial electron flow and ATP synthesis, and induce apoptotic and necrotic death in rapidly growing cells (Miccoli et al., J. Nat. Cancer Inst. 90: 1401-1406, 1998).

Diamide (diazenedicarboxylic acid), the thiol cross-linking non-cyclic amine, completely opens mitochondrial transmembrane pores, which causes the mitochondrial transmembrane potential to collapse, with dissipation of H+ (pH) gradients, production of O2-, and release of the apoptosis inducing factors cytochrome c and AIF. Consequently, cells slowly die depending on their supplies of reduced thiols, primarily glutathione (Zamzami et al., Oncogene 16: 1055-1062, 1998). However, although a potent eradicator of cancer cells and other proliferating cells of the subject, this cross-linking non-cyclic amine is too toxic for clinical uses.

Other cyclic lipophilic amines, such as amino phthalazinediones, biopterins, and rhodamines, which accumulate electrostatically in mitochondrial transmembrane pores and accept and release both electrons and protons, will reversibly serve as both electron and pH buffers in the polarized channels and pores. In this manner, the ionic and oxidative status of the labile sulfhydryl in ANT will be maintained by these redox- and pH-sensitive amines. The cyclic amines will thus affect voltage in the channels, and fluxing electrons are either trapped by O₂ as O₂— or proceed downstream with production of H₂O and ATP. At low doses of these compounds, electron flow will be increased, electrons will proceed downstream to H₂O, ATP production will increase, DNA synthesis and cell proliferation will increase, and cell death is aborted. At high doses, electron flow to H₂O decreases, substrate anion translocations falter, membrane potential declines, ATP production ceases, as does electron flow, and cells go into a quiescent G0/G1 phase or apoptosis.

With the lipophilic tetramethyl-rhodamine, many electrons are shunted directly to O₂, with the result that O₂ accumulates, mitochondrial transmembrane pores open with loss of membrane potential, and apoptotic and necrotic pathways are activated (De Giorgi et al., FASEB J. 10: 607-609, 2002). Phthalazinediones, such as amino derivatives, combined with reduced biopterins, thiols, or lipoic acid, will modulate electron flow to O₂— or H₂O (Lynn et al., unpublished). Specifically, at low doses, amino phthalazinediones will upregulate the subject's immune responses to eradicate cancerous cells. At high doses, amino phthalazinediones stop proliferation of hyperproliferating cancerous cells. Thus, by upregulating or downregulating particular cells, amino phthalazinediones will be useful in cancer treatment (Tzyb et al., Int. J. Immunorehabilitation 12: 398-403, 1999).

Modulation of mitochondria by these bifunctional cyclic phthalazines is most effective in controlling cell fate in proliferating cells that are deficient in biological thiol redox buffers (Armstrong and Jones, FASEB J. 16: [online], Jun. 7, 2002; Larochette et al., Exp. Cell Res. 249: 413-471, 1999), or in proliferating cells deficient in cell cycle checkpoint genes (Yan et al., Genes and Dev., in press). Thus, redox- and pH-sensitive amines that buffer by dually modulating mitochondrial transmembrane pores and anion channels will be clinically useful both in preventing and treating hyperproliferation states such as cancers.

### Example 11: Use of Phthalazinediones in Chronic Dys-Metabolic Syndromes

Food intake, especially fat, with excess deposition of fat in adipose cells causes production and secretion of large amounts of the adipose tissue defense peptide hormones—resistin, leptin, tumor necrosis factor, adiponectin. These collagen- and complement-like peptides facilitate uptake of glucose and combustion of long-chain fatty acids via peroxisome proliferator receptors (PPAR) and mitochondria, with production of heat in the muscle mitochondria, facilitated by activating uncoupling proteins in mitochondria. This removal of the excess fatty acids relieves the fatty acid-induced stress in adipocytes and also lowers levels of toxic, free fatty acids in blood.

However, in time, with prolonged intake of fatty foods, as in affluent societies, and with consequent excessive storage of fat in adipose cells, these overstuffed fat cells produce and secrete more of the inflammatory cytokines, tumor necrosis factor, and resistin (a redox-sensitive adipokine), at the expense of secretion of adiponectin. In aging individuals with overstuffed fat cells, blood levels of tumor necrosis factor and resistin are high; adiponectin and plasminogen-activator inhibitors are low; glucose, free fatty acids, triglyceride, and insulin are high; and the PPARγ/RXR (retinoid X receptor) complexes in fat and muscle cells are under-activated. Vascular accidents in heart and brain, with atherosclerotic plaques, are also greatly increased in these insulin-resistant individuals. This metabolic syndrome, also called Syndrome X, is epidemic.

Metabolic syndrome is a condition marked by excessive abdominal fat, diabetes, high blood pressure, and cholesterol problems, and is caused by the body's inability to use insulin efficiently, which in turn results from overeating and inactivity. Metabolic syndrome is currently and partially treated with various benzolated thiazolidinediones. These cyclic nitrogenous diketones, which are structurally similar to the phthalazinediones of the present invention, bind to the promoters of PPARγ in the nucleus and activate multiple gene families that activate peroxisomal fatty acid oxidation with increased production of adiponectin and catalase, increased glucose uptake, and increased production of enzymes required for fatty acid synthesis and oxidation and for terminal differentiation in adipocytic precursor cells. At high concentrations, these diketone ligands of PPARγ also block proliferation and activities of activated macrophages, endothelial cells, microglia in brain, and probably proliferating smooth muscle cells in atheromatous plaques. Thus, benzolated thiazolidinediones are useful in preventing metabolic syndrome and its downstream sequelae, including insulin resistance, vascular degeneration with hypertension, macrophage proliferation and hyperactivity, with plaque formation and type II diabetes.

Benzolated phthalazinediones chemically resemble benzolated thiazolidinediones and will known to reproduce some functions of benzolated thiazolidinediones, perhaps as a ligand for PPARγ. In particular, amino phthalazinediones, like benzolated thiazolidinediones, will also stop proliferation and suppress destructive overactivity by inflammatory and adipose cells, with production of many inflammogens.

Since benzolated thiazolidinediones are very poor redox agents, it is not likely that they directly modulate thiol redox status in mitochondrial voltage-dependent channels or in the permeability pores. In contrast, since amino phthalazinediones possess these dual defensive functions, as a redox buffer in mitochondria and as a PPAR activator in the nucleus, amino phthalazinediones will provide a better and more complete therapy for all symptoms of metabolic syndrome. Combinational therapy with benzolated thiazolidinediones and amino phthalazinediones, plus thiols and other redox adjuvants, will be the treatment of choice for prevention of downstream sequelae of metabolic syndrome, such as hyperglycemia, hyper fatty acidemia, increased tumor necrosis factor and resistin levels, hypo-adiponectin-emia, hyper or hypo insulin-emia, impaired thiol redox status (hypo-glutathione and cysteine-emia), PPARγ inactivity, and mitochondrial energy uncoupling with elevated H₂O₂, OHOO., and cytoplasmic cytochrome c.

Repeated monitoring of the above adipose hormones during treatments with benzolated thiazolidinediones/amino phthalazinedione/thiol therapies will be required to establish specific dosage and efficacy for each individual. Since with each individual, downstream sequelae of metabolic syndrome, including insulin resistance with long-chain fatty acid poisoning, vary greatly, dose adjustments according to individual responses, as measured by the above adipokine markers, will be required for optimum therapy.

### Example 12: Stress-Induced Phosphorylation Signaling and Phthalazinediones

The major survival and growth signaling pathways in some cells involve the phosphorylation of epidermal growth factor receptor (EGFR), mitogen-activated protein kinases (MAPK), extracellular signal-regulated kinases (ERK), phosphoinositol-3 kinase, protein kinase B, and inhibitor KB kinase (IKK), the kinase controlling NFKB activity, NFKB being a major stress-induced transcription factor. The cell death pathway is controlled by c-Jun N-terminal kinase (JNK), p38, and p53, another stress-induced transcription factor.

Oxidants such as H2O2 activate intracellular phosphorylation cascades responsible for cell survival and growth and for cell death via apoptosis and necrosis (Wang et al., J. Biol. Chem. 275: 14624-14631, 2000). Low doses of H2O2 directly and rapidly activate the survival pathway, using primarily Akt, PI-3K, EGFR, and NFKB. The apoptotic factors Bad and caspase 9 are also downregulated by low doses of H₂O₂. Higher doses of H₂O₂ or prolonged exposure to H₂O₂ activate the cell death pathways involving JNK, p53, Bax, sphingomyelinase, caspases, and the apoptosis signaling kinase AsK-1.

Thus, oxidants, much like the phthalazinediones of the invention, will activate either cell survival or cell death pathways, depending on dosage. However, H₂O₂ is not a buffer and cannot maintain optimal redox potentials sufficient to maintain cell signaling and growth. H₂O₂ also does not scavenge the excess reactive oxygen species produced by activated cell growth pathways. The ability of phthalazinediones, especially amino phthalazinediones, to provide both oxidizing and reducing potential to mitochondria, peroxisomes, and cytoplasmic signaling pathways makes these compounds an ideal in vivo redox buffer capable of dictating most cell fates.

In disease states where signal-induced cell death rates exceed cell growth rates—as in various neurodegenerative syndromes such as Alzheimer's disease, ataxia telangiectasia, Parkinson's disease, multiple system atrophy, or AIDS—or in disease states where autonomous growth signaling rates exceed cell death rates—as in cancers, ataxia telangiectasia, trichothiodystrophy, or hyperinflammatory syndromes—amino phthalazinediones dictate cell fates by buffering the aberrant cellular redox potentials up or down, both in the stressed patient and in any invading or overactivated cell. The phthalazinediones of the invention are therapeutically useful for modulating aberrant phosphorylation signaling syndromes involved in cell growth and death.

### Example 13: Neuronal Overactivity and Amino Phthalazinediones

In Parkinson's disease, neurons of the subthalamic nucleus (STN) become imbalanced and discharge too much. This 4 Hz oscillatory overactivity in STN neurons of patients with the classical symptoms of parkinsonism—bradykinesia, rigidity, and tremor—is a major etiologic factor in Parkinson's disease. Suppression of this oscillatory activity by intra-STN injection of various agents such as lidocaine and muscimol (a gamma aminobutyric acid-A receptor agonist) or chronic electrical (2V) stimulation promptly relieves these parkinsonian symptoms.

The cause of this 4 Hz overactivity in only a few STN neurons is not known (Levy et al., Brain 124: 2105-2118, 2001; Luo et al., Science 298: 425-429, 2002; Limousin et al., New England J. of Med. 339: 1105-1111, 1998; Alvarez et al., Movement Disorders 16: 72-78, 2001). The downstream effects of STN overactivity in substantia nigra reticulata, globus pallidus, and motor thalamus are likely to be responsible for multiple movement disorders.

Since maintaining this excessive and imbalanced 4 Hz oscillation requires increased energy expenditures, agents such as amino phthalazinediones, which can modulate thiol redox status, downregulate mitochondrial energy production, and gain access to the overactivated STN neurons, will suppress the 4 Hz overactivity and thereby suppress and modulate the downstream network activities responsible for the symptoms. Daily intraperitoneal injections of 200 μg of 4-sodium amino phthalazinedione significantly delay the progress of the movement disorder with paralysis induced by MOMU-LV-Ts1 virus in mice. Amino phthalazinediones, however, will suppress both the neuronal and astrocytic overactivity.

### Example 14

The monoisoamyl-2,3-dimercaptosuccinate (MiADMS) ester of 2,3-dimercaptosuccinic acid (DMSSA) can be administered in combination with a phthalazinedione such as the 90% pure monosodium luminol described herein to a subject suffering from cadmium intoxication. Treatment of cadmium intoxicated subjects with MiADMS reversed cadmium induced increase in blood catalase, superoxide dismutase (SOD) and malondialdehyde (MDA), liver MDA and brain SOD and MDA levels but not the decrease in blood, liver brain reduced glutathione (GSH) and increase in oxidized glutathione (GSSG) levels, consistent with the lowering of tissue cadmium burden. The administration of the phthalazinedione will reverse the cadmium induced alteration in the blood and liver GSH, GSSG, blood catalase, SOD, MDA, liver SOD, MDA and brain MDA levels without lowering blood and tissue cadmium contents. However, combined treatment with MiADMS and the phthalazinedione will reverse these alterations as well as reduced blood and tissue cadmium concentrations. The combined treatment will also improve liver and brain endogenous zinc levels, which were decreased due to cadmium toxicity.

### Example 15

Inflammation is one of the key processes underlying metabolic diseases in obese individuals. Large numbers of CD8⁺ effector T cells infiltrated obese epididymal adipose tissue in mice fed a high-fat diet, whereas the numbers of CD4⁺ helper and regulatory T cells were diminished. The infiltration by CD8⁺ T cells preceded the accumulation of macrophages, and immunological and genetic depletion of CD8⁺ T cells lowered macrophage infiltration and adipose tissue infiltration ameliorates systemic insulin resistance. Conversely, adoptive transfer of CD8⁺ T cells to CD8-deficient mice aggravated adipose inflammation. Administration of a pharmaceutical amount of phthalazinedione or phthalazinedione derivative in purified 90% form will modulate the inflammatory response such that the effects of the inflammation are avoided. There is a vicious cycle of interactions between CD8⁺ T cells, macrophages and adipose tissue. Obese adipose tissue activates CD8⁺ T cells, which, in turn, promote the recruitment and activation of macrophages in this tissue.

### Example 16 Helicase Deficiencies

The XPD gene of the xeroderma pigmentosum family codes for a helicase. In XPD deficiency, DNA transcription and repair functions are impaired, resulting in multiple symptoms of early aging (De Boer et al., Science 296: 1276-1281, 2002), Wasting, loss of subcutaneous fat and muscle cells, gray brittle greasy hair with hyperplasia of sebaceous and mammary glands, severe osteoporosis, atrophic germ and stem cells, and immunodegenerative and hyperplastic changes all occur prematurely in XPD-deficient mice or humans.

The failure to maintain normal numbers of cells or normal amounts of cellular thiols, at least in the brittle hair, suggests that a global thiol redox deficiency is responsible for the progressive wasting and chronic cell losses. Since amino derivative phthalazinediones with reduced thiol redox modulators, at low dosage, stimulate cell growth and maintain thiol redox status in cells, treatment with appropriate amounts of reduced thiol redox modulators combined with the phthalazinediones of the present invention will likely prevent the premature aging in regulatory gene deficiencies such as XPD deficiency.

### Example 17 Retroviral-Induced Redox Imbalance

Oncogenie retroviral infections such as HIV in humans or MOMU-LV-Ts1 in mice cause degenerative changes with severe losses of brain cells, immune cells, and germ cells. Other cells like astrocytes and microglia in brain become activated, secrete nitric oxide (NO) and to superoxide (O2-), and grow and accumulate excessively. This imbalance in cell growth and death rates eventually leads to fatal immune and neuronal deficiency syndromes with subsequent transformation in some cells.

In mice infected at 2 days of age with the Ts1 virus, hind limb paralysis occurs with severe wasting, especially of immune organs. In humans infected with HIV, severe immune deficiency with sensory and motor neuropathy also results. In these wasting syndromes with disordered life and death pathways in various cells, some therapeutic attempts with thiol redox modulators other than amino phthalazinediones have been partially successful (Lynn and Wong, Neurolmmunomodulation 4: 277-284 1997; Yan et al., FASEB J. 15: 1132-1138, 2001). In these studies, phthalazinediones plus thiol redox modulators appear to be sufficient to maintain survival and an adequate intracellular thiol redox potential in brain and in thymus.

Since retroviruses activate caspase-dependent apoptosis, and since thiol redox modulators, oxidized and reduced, regulate caspase production from procaspases (Nobel et al., Chem. Res. Toxical. 10: 636-643, 1997), thiol redox modulators in mitochondria, especially an amino phthalazinedione plus dexamethasone, will likely prevent both the loss and the hyperplasia of cells dysregulated by the viruses. Experiments using various thiol redox modulator regimens as preventative therapy in Ts1-infected mice are currently underway.

### Example 18 Polyglutamine Model

In disease states where aberrant peptides slowly accumulate in the brain, early neuronal death with glial hypertrophy occurs. In Huntington's disease, polyglutamine sequences or tracts accumulate in the huntingtin protein. These tracts bind to and inhibit transcription complexes containing Sp-1 and TAFII130 coactivators. Transcription rates decrease, and dysregulated neurons slowly die, first in the caudate nucleus and later in the hippocampus.

Non-proliferating, non-replaceable neurons usually die from metabolic redox imbalances, rather than from programmed death. Therefore, neuronal death in Huntington's disease is likely to be redox-mediated and induced by activation of redox-sensitive cytokines, metalloproteases, and reactive oxygen species (ROS) by activated rnigroglia and astrocytes (Chen et al., Nature Med. 797-801, 2000). In that case, reduced thiol redox-modulators, which at low doses promote cell growth and longevity in redox-suppressed cells, should prove to be useful therapy (Dunah et al., Science 296: 2238-2243, 2002).

In other neurodegenerative syndromes in which aberrant peptides accumulate, including Alzheimer's and Parkinson's diseases, presenilin or synucleins may be responsible for accumulation of the Lewy bodies and β-amyloid peptides. Accumulation of these hydrophobic peptides in plasma, mitochondrial, or endoplasmic reticulum membranes of the cell may be responsible for the neuronal losses in these syndromes. These toxic peptides, like the polyglutamine proteins in Huntington's disease, also lead to astroglia-induced imbalances in thiol redox metabolism, with cell swelling, membrane leakiness, and mitochondrial necrosis. Maintenance of thiol redox status with reduced thiol redox modulators, especially an amino phthatazinedione and acetyl cysteine, should prevent or delay the neuronal death in these degenerative diseases (Wolfe and Selkoe, Science 296: 2156-2157, 2002; Welhofen et al., Science 296: 2215-2218, 2002).

### Example 19 Premature Aging with Cancer Models

In regulatory gene-dependent syndromes of premature aging, including ataxia telangiectasia, Down syndrome, trichothiodystrophy, Bloom's disease, p53 over-activity (De Boer et al., Science 296: 12761281, 2002; Tyner et al., Nature 415: 45-50, 2002), in which life and death of specific cell types are aberrant, appropriate treatments in vitro and in vivo with thiol redox modulators have been partially successful. In ataxia telangiectasia gene (ATM) deficiency in mice, early pretreatment with dexamethasone, the glutathione secretagogue, completely prevents the excessive proliferation and development of the fatal thymic cancer.

Other thiol redox modulators, such as N-acetyl cysteine and dehydrolipoic acid, also delay the premature degeneration of cells and the thymomas. Thiol redox modulators also correct the delayed differentiation and excessive production of DNA in ATM-deficient lymphoid cells (Yan et al., FASEB J. 15: 1132-1138, 2001; Lynn et al., unpublished). However, in the ATM-deficient mice, treatment was fully successful only if the thiol redox modulators were applied early, before two weeks of age and before tumor development.

Dexamethasone alone completely prevents tumor formation if given to 10-day old ATM-deficient mice for three weeks, but does not suppress tumor growth or increase longevity if given at physiologic doses at three months of age. Whether amino phthalazinediones with other thiol redox modulators, which suppress growth of non-transformed ATM-deficient cells in vitro, can fully suppress tumors in vivo, without toxicity, has not been rigorously evaluated. Cross-linking redox modulators such as diamide, menadione, and oxidized phthalazines are known to stop cell growth, activate caspases, and initiate apoptosis in some tumor cells (Pias and Aw, FASEB J. 16: 781-790, 2002).

### Example 20 Neuronal Overactivity and Amino Phthalazinediones

In Parkinson's disease, neurons of the subthalamic nucleus (STN) become imbalanced and discharge too much. This 4 Hz oscillatory overactivity in STN neurons of patients with the classical symptoms of parkinsonism—bradykinesia, rigidity, and tremor—is a major etiologic factor in Parkinson's disease. Suppression of this oscillatory activity by intra-STN injection of various agents such as lidocaine and muscimol (a gamma aminobutyric acid-A receptor agonist) or chronic electrical (2V) stimulation promptly relieves these parkinsonian symptoms.

The cause of this 4 Hz overactivity in only a few STN neurons is not known (Levy et al., Brain 124: 2105-2118, 2001; Luo et al., Science 298: 425-429, 2002; Limousin et al., New England J. of Med. 339: 1105-1111, 1998; Alvarez et al., Movement Disorders 16: 72-78, 2001). The downstream effects of STN overactivity in substantia nigra reticulata, globus pallidus, and motor thalamus are likely to be responsible for multiple movement disorders.

Since maintaining this excessive and imbalanced 4 Hz oscillation requires increased enemy expenditures, agents such as amino phthalazinediones, which can modulate thiol redox status, downregulate mitochondrial energy production, and gain access to the overactivated STN neurons, can potentially suppress the 4 Hz overactivity and thereby suppress and modulate the downstream network activities responsible for the symptoms. Daily intraperitoneal injections of 200 μg of 4-sodium amino phthalazinedione significantly delay the progress of the movement disorder with paralysis induced by MOMU-LV-Ts1 virus in mice. Whether this is due to suppression of oscillatory activity in neurons, suppression of virus-induced astrocytic inflammatory responses, or both is under investigation. Amino phthalazinediones should, however, suppress both the neuronal and astrocytic overactivity.

### Example 21 Multiple Chemical Sensitivity Syndrome

Multiple chemical sensitivity (MCS) is a decompensating syndrome in which individuals develop hypersensitivities to multiple environmental toxins, e.g., exhaust fumes from vehicles, factories, garbage dumps, or explosives, perfumes, sulfur oxides, nitrous oxide, and cyclic hydrocarbons produced by molds or plants. Inhalation of such toxins causes symptoms like fatigue, weakness, loss of equilibrium, sensory impairments in smell, taste, hearing, vision, and sensation, cognitive impairments in memory, concentration, and motivation, and motor symptoms that vary from muscle and hone wasting to athetoid, epileptiform, or fibrillary movements. External signs of premature aging, e.g., graying or loss of hair, wrinkling of skin, bradykinesia, or signs of Syndrome X (type II diabetes, hypertension, hyperlipidemia, atrial fibrillation) are usually present at least to some extent.

The multiple incapacitating symptoms of MCS are rapidly induced by inhaling trace amounts of the noxious substance. Removal of the inciting inhalant prevents the acute symptomology but does not eliminate the hypersensitivity. The most characteristic symptom is an inability to “get up and go,” or lead a productive life. The symptoms may become permanent or persist for months unless the chronic exposure is eliminated. The inhaled toxins are thought to induce redox imbalances in the naso-olfactory system and brain, and preliminary findings suggest that the functional impairments in MCS are due to acute imbalances in NO/O2- and thiol redox potentials in the naso-olfactory system.

Studies also suggest that the neuromuscular and cognitive dysfunctions in MCS may represent redox imbalances in pathways controlling neurotransmission. The constant neurotransmission in neurons, with its repetitive separation of charges, obligatorily produces large amounts of reactive oxygen and nitrogen species. Under stress, these radicals may accumulate with pathologic results. The cell's major redox buffers for controlling these signaling radicals, e.g., reduced thiols such as glutathione, cysteine, and thioredoxins, are rapidly consumed under redox stresses such as anoxia, nutrient or ion deprivation, and aberrant peptide accumulation.

The symptomatic redox imbalances may be corrected by appropriate redox support to the impaired naso-olfactory system. Treatment with thiol redox support, i.e., inhaled or intramuscular glutathione, has been partially successful. Intranasal inhalation of a solution of 25 mg/ml of a phthalazinedione of the invention and 12 mg/ml of sodium glutathione in isotonic NaCl four times per day for three days should remove the incapacitating weakness and the sensory and cognitive symptoms within 48 hours. Inhalation of the reduced phthalazinedione, along with a reduced thiol, can quickly but temporarily rebalance the thiol redox imbalance in the patient's brain and quickly ameliorate some of the symptoms. Motor symptoms may be reversed by prolonged treatment with higher doses of this combined redox therapy.

### Example 22: Manufacturing Phthalazinedione Derivatives

Phthalazinedione derivatives, N-ethanoyl-5-amino-2,3-dihydrophthalazine-1,4-dione and 5-(hexanoyl oxyamino)-2,3-dihydrophthalazine-1,4-dione are manufactured utilizing a Water for Injection (WFI) system that incorporates a pre-treatment system, a vapor compression still, and a WFI distribution loop. Water treatment steps include KDF® filtration for chlorine/chloramines destruction, a polisher cation exchange system for the removal of ammonia, and a MECO® vapor compression pharmaceutical distillation unit for chemical and microbial purification. WFI is charged to reactor A followed by sodium hydroxide pellets. 3-Nitrophthalhydrazide is charged to the clear solution in reactor A to form a slurry that is dark brown in color. The contents of reactor A are dropped out into a mobile vessel and transferred to the Biazzi hydrogenator. The solution is hydrogenated with 5% Pd/C Johnson Matthey type 87L catalyst or equivalent at 40° C. until hydrogen uptake is complete. In Process Control (IPC) is taken to confirm that the hydrogenation is complete. The catalyst is removed by filtration and the filtrate concentrated to approximately 25% of its original volume by distillation. The catalyst utilized in the manufacturing process is used to attach the functional organic groups to the monosodium luminol.

The solution is cooled to 0-6° C. and held for 36-48 hours (depending on the batch size) for crystallization to take place. The product is filtered off and washed. An IPC is taken to ensure that the pH of the product is within specification. If required, the product is subject to additional washes until the pH is within the specified range. The product is partially dried (60-90 min), sieved and dried under vacuum to constant weight (LOD<0.5%). The final product is sieved through a 1 mm mesh. The expected yield is 68-85%. A number of laboratory-scale experiments were performed to optimize reaction conditions and yield, and to achieve consistency.

Concentrations of solutions were altered to optimize plant manipulation. The hydrogenation conditions were altered to allow fast reaction time and minimize dangerous intermediates. Hold-points and expected manipulation times were placed to reduce product colorization and increase product quantity and quality. In addition, the aqueous wash and ethanol re-slurry were removed and replaced by multiple ethanol washes to cut down production costs. Product color was removed by CUNO filtration instead of using powdered charcoal, increasing ease of booth cleaning and reducing hazardous manipulations. Further, the drying method was altered to prevent the product forming large granules and to reduce the chances of the product being fully dried prematurely.

The foregoing material describes various aspects of the invention and how it may be practiced. The description is not intended to be exhaustive of the many different embodiments of the invention Although the foregoing invention has been described in some detail by way of illustration and example, to aid understanding, it will be readily apparent to those of ordinary skill in the art, in light of the teachings of this invention, that certain changes and modifications may be made to the invention without departing from the spirit or scope of the appended claims.

What is claimed is:

1. A phthalazinedione compound comprising N-ethanoyl-5-amino-2,3-dihydrophthalazine-1,4-dione or 5-(hexanoyl oxyamino)-2,3-dihydrophthalazine-1,4-dione.

2. A pharmaceutical composition comprising N-ethanoyl-5-amino-2,3-dihydrophthalazine-1,4-dione or its pharmaceutically acceptable salt, ester, solvate, hydrate, metabolite, enantiomer, isomer, tautomer, amide, prodrug, or free base; and one or more pharmaceutically acceptable carriers.

3. The pharmaceutical composition of claim 2, further comprising an amino acid, antibiotic, antiviral agent, anti-inflammatory agent, antioxidant, immunomodulator, reductant, oxidative protector, steroid, vitamin, or a combination thereof.

4. The pharmaceutical composition of claim 2, wherein the composition comprises a pharmaceutically acceptable form selected from the group consisting of tablet, capsule, granule, powder, solution, suspension, microsphere, liposome, colloid, lyophilized composition, gel, lotion, ointment, cream, spray, and suppository.

5. A method of administering to a subject the pharmaceutical composition of claim 2.

6. The method of claim 5, wherein the pharmaceutical composition is administered to the subject by a means selected from the group consisting of intravenous, intramuscular, intraperitoneal, subcutaneous, oral, nasal, mucosal, transdermal, parenteral, vaginal, and rectal.

7. The method of claim 5, wherein the pharmaceutical composition is administered to the subject in combination with a standard treatment selected from the group consisting of radiation, chemotherapy, laser therapy, surgery, medication, and nutrition.

8. The method of claim 5, wherein the pharmaceutical composition is administered to the subject to treat metabolic distress, inflammatory conditions, infectious conditions, neurological disorders, immune disorders, proliferative diseases, or cellular senescence.

9. The method of claim 5, wherein the pharmaceutical composition is administered to the subject in a dose comprising the N-ethanoyl-5-amino-2,3-dihydrophthalazine-1,4-dione in an amount of about 0.01 mg/kg to about 100.0 mg/kg of body weight.

10. The method of claim 5, wherein the pharmaceutical composition is administered to the subject in a dose comprising the N-ethanoyl-5-amino-2,3-dihydrophthalazine-1,4-dione in an amount of about 1.0 mg per day to about 10,000.0 mg per day.

11. A pharmaceutical composition comprising 5-(hexanoyl oxyamino)-2,3-dihydrophthalazine-1,4-dione or its pharmaceutically acceptable salt, ester, solvate, hydrate, metabolite, enantiomer, isomer, tautomer, amide, prodrug, or free base; and one or more pharmaceutically acceptable carriers.

12. The pharmaceutical composition of claim 11, further comprising an amino acid, antibiotic, antiviral agent, anti-inflammatory agent, antioxidant, immunomodulator, reductant, oxidative protector, steroid, vitamin, or a combination thereof.

13. The pharmaceutical composition of claim 11, wherein the composition comprises a pharmaceutically acceptable form selected from the group consisting of tablet, capsule, granule, powder, solution, suspension, microsphere, liposome, colloid, lyophilized composition, gel, lotion, ointment, cream, spray, and suppository.

14. A method of administering to a subject the pharmaceutical composition of claim 11.

15. The method of claim 14, wherein the pharmaceutical composition is administered by a means selected from the group consisting of intravenous, intramuscular, intraperitoneal, subcutaneous, oral, nasal, mucosal, transdermal, parenteral, vaginal, and rectal.

16. The method of claim 14, wherein the pharmaceutical composition administered is used in combination with a standard treatment selected from the group consisting of radiation, chemotherapy, laser therapy, surgery, medication, and nutrition.

17. The method of claim 14, wherein the pharmaceutical composition is administered to the subject to treat metabolic distress, inflammatory conditions, infectious conditions, neurological disorders, immune disorders, proliferative diseases, or cellular senescence.

18. The method of claim 14, wherein the pharmaceutical composition is administered to the subject in a dose comprising the 5-(hexanoyl oxyamino)-2,3-dihydrophthalazine-1,4-dione in an amount of about 0.01 mg/kg to about 100.0 mg/kg of body weight.

19. The method of claim 14, wherein the pharmaceutical composition is administered to the subject in a dose comprising the 5-(hexanoyl oxyamino)-2,3-dihydrophthalazine-1,4-dione in an amount of about 1.0 mg per day to about 10,000.0 mg per day.
